(19) United States
(12) Patent Application Publication
(10) Pub. No.: US 2026/0356882 A1
(43) Pub. Date: Nov. 12, 2026

(54) METHODS FOR PREPARING PROTEIN-CONJUGATED NANOCARRIERS

(71) Applicant: Northwestern University, Evanston, IL (US)

(72) Inventors: Justin Alexander Peruzzi, Evanston, IL (US); Neha Prashant Kamat, Evanston, IL (US); Timothy Quang Vu, Evanston, IL (US)

(21) Appl. No.: 19/120,001

(22) PCT Filed: Oct. 10, 2023
(86) PCT No.: PCT/US2023/076459

§ 371 Date: Apr. 10, 2025

Related U.S. Application Data
(60) Provisional application No. 63/378,989, filed on Oct. 10, 2022.
(60) Provisional application No. 63/492,152, filed on Mar. 24, 2023.

(51) Int. Cl. C07K 1/107; A61K 47/64; A61K 47/69

(52) U.S. Cl. CPC C07K 1/1077; A61K 47/64; A61K 47/6913; C07K 2319/40

(57) ABSTRACT

Provided herein are methods for functionalizing nanoparticles with functional proteins, and methods of using the functionalized nanoparticles. Compositions for preparing the functionalized nanoparticles are also described herein.

### CROSS-REFERENCE TO RELATED APPLICATIONS

This application claims priority to U.S. Provisional Application Nos. 63/378,989 filed on Oct. 10, 2022, and 63/492,152 filed on Mar. 24, 2023, the contents of which are incorporated by reference in their entireties.

### STATEMENT REGARDING FEDERALLY SPONSORED RESEARCH

This invention was made with government support under CBET-1844336 awarded by the National Science Foundation. The government has certain rights in the invention.

### REFERENCE TO AN ELECTRONIC SEQUENCE LISTING

The contents of the electronic sequence listing (702581.02415.xml; Size: 19,646 bytes; and Date of Creation: Oct. 10, 2023) is herein incorporated by reference in its entirety.

### BACKGROUND OF THE INVENTION

Over the last 20 years, there has been an explosion in the development of biomolecular tools, ranging from small molecule drugs to proteins, which can alter cell state. Such molecules have great promise for treating diseases such as cancer, however we are still limited by the ability to alter the molecular pharmacokinetics and specificity in a generalizable manner. Previously, nanocarriers such as liposomes have been used to deliver chemotherapies in order to reduce toxicity. More recently, lipid nanocarriers have proven to be effective vaccines to fight the COVID-19 pandemic. However, these therapeutic systems still lack the ability to specifically deliver cargo and evade the body's surveillance, resulting in rapid clearance.

The adoption of protein-functionalized, cell-mimetic nanocarriers in the clinic has been slow as producing, purifying, and conjugating recombinant proteins to membrane-based materials is a costly and time intensive process. Traditional protein production methods require recombinant protein expression and purification, which can take on the order of days to weeks, and often requires optimization for each protein. Subsequent steps to attach the proteins to nanocarriers provide further obstacles: active conjugation strategies are typically not site-selective and low-efficiency conjugation methods result in poor yields and wasted protein.^{[5]} These shortcomings in the production of protein-modified nanocarriers make developing and testing protein functionalized cell-mimetic materials slow and difficult to produce.

Thus, there exists a need in the art for methods to rapidly design, express and conjugate proteins to the surface of membrane-based nanocarriers.

### SUMMARY OF THE INVENTION

In an aspect, provided herein is a method for functionalizing a nanocarrier with a functional protein, the method comprising assembling the nanocarrier with a modified lipid; incubating the assembled nanocarrier with a cell-free protein synthesis system and a nucleic acid template encoding a fusion protein, the fusion protein comprising a protein tag that conjugates to the modified lipid; and the functional protein. In embodiments, the protein tag is a self-labeling enzyme. In embodiments, the modified lipid comprises benzylguanine and the self-labeling enzyme is a SNAP-tag. In embodiments, the functional protein is selected from an antibody fragment, a de novo designed protein, and a receptor. In embodiments, the nanocarrier is a liposome, a polymersome, a lipid nanoparticle, an extracellular vesicle, or an outer membrane vesicle.

In another aspect, provided herein is a method for functionalizing a nanocarrier with a functional protein, the method comprising incubating the nanocarrier with a cell-free protein synthesis system and a nucleic acid template encoding a fusion protein, the fusion protein comprising a transmembrane domain; and the functional protein. In embodiments, the functional protein is selected from an antibody fragment, a de novo designed protein, and a receptor.

In another aspect, provided herein is a method for delivering a cargo to a cell, the method comprising encapsulating the cargo into a nanocarrier; functionalizing the nanocarrier according to any of the methods described herein; and dosing the cell with the functionalized nanocarrier; wherein the cell comprises a surface protein that binds to the functional protein. In embodiments, the cargo comprises a polynucleotide, a polypeptide, an active pharmaceutical ingredient, or a therapeutic agent. In embodiments, the cargo is an mRNA molecule.

In another aspect, provided herein is a method for generating an artificial antigen presenting cell, the method comprising functionalizing a nanocarrier according to any of the methods described herein, wherein the functional protein is an antigen. In embodiments, the antigen is an scFv. In embodiments, the scFv is specific for T lymphocytes. In embodiments, the nanocarrier comprises one or more additional nucleic acid templates, wherein each nucleic acid template of the nanocarrier encodes a different functional protein.

In another aspect, provided herein is a method for activating a T lymphocyte, the method comprising incubating the T lymphocyte with any of the artificial antigen presenting cells described herein.

In another aspect, provided herein is a composition comprising a nanocarrier comprising a modified lipid; a cell-free protein synthesis system; and a nucleic acid template encoding a fusion protein, the fusion protein comprising a protein tag that conjugates to the modified lipid; and a functional protein. In embodiments, the protein tag is a self-labeling enzyme. In embodiments, the modified lipid comprises benzylguanine and the self-labeling enzyme is a SNAP-tag. In embodiments, the functional protein is selected from an antibody fragment, a de novo designed protein, and a receptor. In embodiments, the nanocarrier is a liposome, a polymersome, or a lipid nanoparticle. In embodiments, the nanocarrier further comprises a cargo molecule. In embodiments, the cargo molecule comprises a polynucleotide, a polypeptide, an active pharmaceutical ingredient, or a therapeutic agent.

In another aspect, provided herein is a composition comprising a nanocarrier; a cell-free protein synthesis system; and a nucleic acid template encoding a fusion protein, the fusion protein comprising a transmembrane domain; and a functional protein. In embodiments, the functional protein is selected from an antibody fragment, a de novo designed protein, and a receptor. In embodiments, the nanocarrier is a liposome, a polymersome, or a lipid nanoparticle. In embodiments, the nanocarrier further comprises a cargo molecule. In embodiments, the cargo molecule comprises a polynucleotide, a polypeptide, an active pharmaceutical ingredient, or a therapeutic agent.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1E. Cell-free expressed proteins can be conjugated to liposomes. A) Proteins can be expressed and conjugated to liposomes via a self-labeling enzyme. By adding 0.1 mol % 18:1 PE PEG2000 benzylguanine to the membrane and fusing a SNAP tag to mEGFP, SNAP-mEGFP forms a covalent bond with the benzylguanine labeled membrane. (B, C) SNAP-mEGFP conjugation to liposomes is confirmed via size exclusion chromatography. mEGFP elutes with liposomes when fused to a SNAP tag and 18:1 PE PEG2000 benzylguanine lipid is present in liposome membranes (vesicle fraction) and free mEGFP elutes later (free fraction). D) 62% of the benzylguanine lipid is bound to mEGFP after conjugation, or a ratio of 6E-4 mol protein to 1 mol of lipid, as determined by comparing Rhodamine lipid and mEGFP fluorescence in elution fractions to known standards. E) Liposome diameter before and after mEGFP conjugation, as measured by dynamic light scattering. Liposomes were composed of 68.9 mol % DOPC, 30 mol % cholesterol, 0.9 mol % 18:0 PEG2000, 0.1 mol % 18:1 PE PEG2000 benzylguanine, and 0.1 mol % 18:1 Rhodamine PE unless otherwise specified.

FIGS. 2A-2E. Cell-free expressed proteins can bind and localize lipid-based particles to cells. Binding of A) mEGFP, B) αHER2 affibody, C) computationally designed αCD3 miniprotein, and D) αCD3 scFv to Jurkat, MDA-MB-231, and SKBR3 cells in soluble and liposome conjugated forms was assessed. Soluble proteins were labeled with fluorophore SNAP547 and liposomes were labeled with lipid conjugated rhodamine. Binding of proteins was assessed via flow cytometry. Reported is the fold change in fluorescence of each condition relative to the fluorescence of unstained cells. E) Ratio of fold change of vesicle bound protein to soluble protein shows vesicle conjugation enhances binding. Conditions which should be positive for protein binding are outlined. Liposomes were composed of 68.9 mol % DOPC, 30 mol % cholesterol, 0.9 mol % 18:0 PEG2000, 0.1 mol % 18:1 PE PEG2000 benzylguanine, and 0.1 mol % 18:1 Rhodamine PE. All experiments were performed n=3, error bars represent the S.E.M.

FIGS. 3A-3C. Functionalization is indiscriminate to functionalization strategy and particle type. SNAP-αHER2 affibody was conjugated to A) LNPs and B) polymersome via 18:1 PE PEG2000 benzylguanine and their binding to HER2 expressing SKBR3 cells was assessed via flow cytometry. C) The αHER2 affibody can be functionalized to liposomes via transmembrane domain. Plots report the fold change in median MFI over an unstained control. Polymersomes were composed of 99.8 mol % poly(ethylene oxide)-b-poly butadiene, 0.1 mol % 18:1 PE PEG2000 benzylguanine, and mol % 18:1 rhodamine. LNPs were composed of 50 mol % D-Lin-MC3-DMA, 10 mol % DSPC, 38 mol % cholesterol, 0.9 mol % 18:0 PEG2000, 0.1 mol % 18:1 PE PEG2000 benzylguanine, and 0.1 mol % 18:1 Rhodamine PE. Liposomes used in transmembrane domain conjugation experiments were composed of 69.9 mol % DOPC, 30 mol % cholesterol, and 0.1 mol % 18:1 Rhodamine PE. All experiments were performed n=3, error bars represent the S.E.M.

FIGS. 4A-4E. Protein functionalized liposomes as artificial antigen presenting cells (aAPCs) to activate T-cells. A) A T-cell line which expresses GFP upon activation was used to assess cell-free protein conjugated liposomes ability to interact with and activate T-cells. B) NFAT GFP-Jurkat cells express GFP when incubated with liposomes conjugated to αCD3 scFv but not when incubated with only liposomes or only protein. Increasing the C) liposome dose and D) the amount of protein conjugated to liposomes enhances the amount of GFP expressed. The farthest point on the right corresponds to condition in (B). E) Functionalizing liposomes with αCD3 and αCD2 scFv enhances activation above αCD3 alone. All experiments were performed n=3, error bars represent the S.E.M.

FIGS. 5A-5C. Protein functionalized lipid nanoparticles can deliver mRNA cargo to T-cells. A) The ability of lipid nanoparticles functionalized with cell-free expressed proteins to functionally deliver mRNA to T cells was assessed. B) Microscopy images of T cell treated with lipid nanoparticles encapsulating eGFP functionalized with no protein or αCD3 scFv after 24 hours. Scale bars are 100 μm. C) Lipid nanoparticles encapsulating Fluc were then functionalized with no protein, αCD3 scFv, αCD2 scFv, αCD3 scFv and αCD2 scFv, or αCD3 scFv and αHER2 affibody. Luciferase expression, as a result of mRNA delivery, was assessed 24 hours after dosing Jurkat cells with 1000 ng of mRNA. Lipid nanoparticles were composed of 50 mol % D-Lin-MC3-DMA, 10 mol % DSPC, 38 mol % cholesterol, 0.9 mol % 18:0 PEG2000, and 0.1 mol % 18:1 PE PEG2000 benzylguanine. Experiments were performed n=3, error bars represent the S.E.M.

FIG. 6. Cell-free expressed proteins can enhance the functionality of membrane-based materials. Cell-free expressed protein and membrane-based nanoparticles can be produced in parallel and then conjugated to produce functionalized nanoparticles within a single day. These nanoparticles are amenable to a wide array of downstream therapeutic applications.

FIG. 7. mEGFP and vesicle quantification using lipid and protein standards. Fluorescence standard curves for liposomes labeled with a lipid conjugated rhodamine (top) and purified GFP (bottom) were generated and compared to fluorescence of purified mEGFP conjugated liposomes (shown in FIG. 1). Using standard curves, the amount of lipid and protein was quantified (blue points) and the ratio of GFP to lipid was calculated.

FIGS. 8A-8B. Expression and quantification of binding proteins. A) Chimeric SNAP proteins were labeled with SNAP547 and run on a denaturing SDS-PAGE gel. B) Protein band intensity was quantified and compared to purified protein standard to determine protein concentrations. Protein expression was performed n=3, error bars represent the S. E. M.

FIG. 9. Expression of surface proteins by commercial antibodies. Binding of proteins was assessed via flow cytometry. Reported is the fold change in fluorescence of each condition relative to the fluorescence of unstained cells.

FIG. 10. Gating strategy and flow cytometry for soluble and vesicle-bound protein binding. Cells were first gated by size through forward and side scatter, then gated by singlets.

FIGS. 11A-11B. Control vesicles without benzylguanine lipid mixed with cell free protein binders do not bind to cells, as shown with A) flow cytometry and B) a heat map. Reported is the fold change in fluorescence of each condition relative to the fluorescence of unstained cells.

FIG. 12. Dose-dependent binding of anti-HER2 vesicles and unconjugated, control vesicles to SKBR3 cells. Hook effect starts to occur at concentrations above 100 μM of lipid vesicles. Error bars=standard deviation, n=2.

FIGS. 13A-13B. Expanded panel of binding proteins tested in this study. A) Chimeric SNAP proteins were labeled with SNAP547 and binding to cells expressing protein was assessed via flow cytometry. B) Proteins were then conjugated to liposomes composed of 68.9 mol % DOPC, 30 mol % cholesterol, 0.9 mol % 18:0 PEG2000, 0.1 mol % 18:1 PE PEG2000 benzylguanine, and 0.1 mol % 18:1 Rhodamine PE. Liposome binding to cells was then assessed via flow cytometry. All experiments were performed n=3.

FIG. 14. Titration for transmembrane αHER2 affibody functionalized liposomes. Transmembrane fused αHER2 affibody was cotranslationally inserted into liposomes composed of 69.9 mol % DOPC, 30 mol % cholesterol, and 0.1 mol % 18:1 Rhodamine PE. Liposome binding to SKBR3 cells was assessed via flow cytometry. All experiments were performed n=3, error bars represent the S.E.M.

FIG. 15. Comparison of uniform (top) and phase-separated (bottom) αCD3 vesicles for vesicle binding and activation of NFAT-Jurkat cells. All experiments were performed n=2, error bars represent the S.E.M.

FIG. 16. Transfection of Jurkat cells with GFP-mRNA using LNPs with no protein or conjugated to αCD3 after 24 hours with 2000 ng RNA detected by flow cytometry.

FIGS. 17A-17C. Delivery of Fluc mRNA to T cells via protein functionalized lipid nanoparticles. (A) The encapsulation efficiency of Fluc mRNA in lipid nanoparticles was quantified using a RiboGreen assay. Lipid nanoparticles were then with αCD3 scFv, αCD2 scFv, αCD3 scFv and αCD2 scFv, or αCD3 scFv and αHER2 affibody (a nonbinder) and dosed to 100,000 Jurkat cells. After 24 hours, (B) Fluc expression was measured via luminesence and (C) cell viability was confirmed. All experiments were performed n=3, error bars represent the S.E.M.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The surface modification of membrane-based nanocarriers, such as liposomes, polymersomes, lipid nanoparticles, extracellular vesicles, and outer-membrane vesicles with targeting molecules is an important step in the design of therapeutic materials. However, this modification can be costly or time-consuming, requiring cellular hosts for protein expression, and lengthy purification and conjugation steps to attach the proteins to the surface of nanocarriers, and ultimately limits the development of effective protein conjugated nanocarriers. Demonstrated herein is the use of cell-free protein synthesis systems to rapidly create protein-conjugated membrane-based nanocarriers. Using this approach, multiple types of functional binding proteins, including affibodies, computationally designed proteins, as well as scFvs, can be expressed cell-free and conjugated to liposomes in one-pot. This technique can be further expanded to other nanocarriers, including polymersomes and lipid nanoparticles, and is amenable to multiple conjugation strategies, including surface attachment to and integration into nanocarrier membranes. These methods are further leveraged in vitro demonstrating rapidly designed bispecific artificial antigen presenting cells and enhanced delivery of lipid nanocarrier cargo. This workflow enables the rapid generation of membrane-based delivery systems and bolster the ability to create cell-mimetic therapeutics.

As described further herein, cell-free expressed proteins may be conjugated to lipid-based nanocarriers. Conjugation of proteins may be through chemical conjugation, for example chemical or self-labeling conjugation or via hydrophobic transmembrane domain insertion. Different conjugation methods may allow for a protein of interest to be conjugated to a lipid-based nanocarrier in a site-specific manner. Chemical conjugation may allow for a protein to be conjugated to the surface of lipid-based nanocarriers. Conjugation with a transmembrane domain allows for a protein to be inserted into the membrane of lipid-based nanocarriers.

In a first aspect, provided herein is a method for functionalizing a nanocarrier, the method comprising assembling the nanocarrier with a modified lipid, incubating the nanocarrier with a nucleic acid template encoding a fusion protein and a cell-free protein synthesis system, the fusion protein comprising a protein tag that conjugates to the modified lipid; and the functional protein.

The term “nanocarrier” refers to a nanomaterial used as a transport module for another substance. Nanocarriers include, without limitation, liposomes, micelles, nanoparticles, extracellular vesicles (e.g. vesicles derived from mammalian cells), outer membrane vesicles (e.g. vesicles derived from bacteria), and dendrimers. The methods described herein may also be used to functionalize microparticles and other therapeutic materials, such as giant unilamellar vesicles, inorganic beads, hydrogels, and cell membranes.

Lipid-based nanocarriers are colloidal carriers for bioactive organic molecules. A “lipid layer,” “lipid structure,” or “lipid membrane” means a continuous, self-assembled barrier comprising a plurality of amphiphilic lipids. In some embodiments, the lipid layer comprises a single layer of amphiphilic lipids, e.g., a micelle or a reverse micelle, having a hydrophilic surface and a hydrophobic surface. In other embodiments, the lipid layer is a lipid bilayer comprising two layers of amphiphilic lipids having an inner-hydrophilic surface, an outer-hydrophilic surface, and a hydrophobic core disposed between the inner-hydrophilic surface and the outer-hydrophilic surface, e.g., a liposome, a lipid nanoparticle, a cell, a cellular organelle, or a 2-dimensional membrane.

“Amphiphilic lipid” means any chemical compound having both hydrophilic and hydrophobic properties and typically composed of a polar head group and lipophilic tail. The polar head group may charged or uncharged. Suitably, the polar head groups may comprise anionic head groups (such as carboxylates, sulfates, sulfonates, or phosphates), cationic head groups (such as ammoniums), or uncharged head groups (such as alcohols). The lipophilic tail is typically a saturated or unsaturated alkyl or a saturated or unsaturated alkylene having at least four carbon atoms, suitably between 6 and 24 carbon atoms. Exemplary amphiphilic lipids include, without limitation, phospholipids (e.g., sphingomyelins or phosphoglycerides such as phosphatidylserines, phosphatidylethanolamines, phosphatidylinositols, or phosphatidylcholines), glycolipids, fatty acids, amphiphilic di-block copolymers, amphiphilic tri-block copolymers, amphiphilic dendrimers, amphiphilic dendrons, or peptide amphiphiles.

The term “functionalize” refers to adding a functional protein to the surface of a nanocarrier. Functionalizing the nanocarrier may result in specific recognition of the nanocarrier by other proteins in the environment. Accordingly, a “functional protein” may be any protein that is displayed on the surface of the nanocarrier, and results in specific recognition of the nanocarrier by other proteins in the environment. Functional proteins include, but are not limited to, antibodies, antibody fragments, (e.g. affibodies, Fab, Fab′, F(ab′)2, scFv, Fv, dsFv, ds-scFv, Fd, dAbs, TandAbs dimers, mini bodies, monobodies, diabodies, nanobodies, and multimers thereof, etc.), de novo or computationally designed proteins, receptors, and miniproteins. The functional protein may have biological activity. The functional protein may be a therapeutic agent. The functional protein may bind to a cellular protein, such as a cell surface protein. The functional protein may be bispecific. A bispecific protein can bind two different antigens or epitopes at the same time. The different antigens or epitopes may be on the same or different cells. Functional proteins also include proteins that label the nanocarrier, such as fluorescent proteins or markers.

The nanocarriers disclosed herein may be assembled or prepared using polymer materials including modified lipids. A modified lipid may be used as a substrate to bind a functional protein to the nanocarrier surface. In exemplary embodiments, the nanocarriers are assembled with O⁶-benzylguanine (BG) or BG-derivatives. BG and BG-derivates that serve as substrates for SNAP-tag may be used. Other modified lipids may be incorporated into the nanocarrier to avoid detection or the nanocarrier by the immune system, to alter the solubility of the nanocarrier, to alter the bioavailability of the nanocarrier, etc.

Once assembled, the nanocarrier is incubated with a cell-free protein synthesis system including the components necessary for transcription and translation of a protein; and a nucleic acid template encoding a fusion protein and having the regulatory components required for protein transcription and translation, such as a promoter and ribosome binding site.

A “cell-free system” or “CFPS reaction mixture” typically contains a crude or partially-purified cell extract, an RNA translation template, and a suitable reaction buffer for promoting cell-free protein synthesis from the RNA translation template. The cell-free system may also, or alternatively, include a DNA expression template encoding an open reading frame operably linked to a promoter element for a DNA-dependent RNA polymerase. Additional NTP's and divalent cation cofactor may be included in the cell-free system. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The cell-free systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

The cell-free system may comprise a cellular extract from a host strain. Because cell-free protein synthesis (CFPS) systems exploit an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Patent Application Publication No. 2014/0295492 and U.S. Patent Application Publication No. 2016/0060301, the contents of which are incorporated by reference in their entireties. The cellular extract of the platform may be prepared from a cell culture of a prokaryote (e.g., E. coli). While E. coli is exemplified herein, the bacterial species is not intended to be limiting. Other bacterial species suitable for the compositions and methods disclosed herein include but are not limited to (e.g., bacillis species such as Bacillus subtilis, Vibrio species such as Vibrio natriegens, Pseudomonas species, etc.). In some embodiments, the cell culture is in stationary phase. In some embodiments, stationary phase may be defined as the cell culture having an OD600 of greater than about 3.0, 3.5, 4.0, 4.5, 5.0, 5.5, 6.0, 6.5, 7.0, 7.5, 8.0, or having an OD600 within a range bounded by any of these values. Further methods for preparing a cell-free system are disclosed in International Patent Application Publication No. WO2020185451A2, the contents of which are incorporated by reference in its entirety.

The cell extract may be prepared by lysing the cells of the cell culture and isolating a fraction from the lysed cells. For example, the cell extract may be prepared by lysing the cells of the cell culture and subjecting the lysed cells to centrifugal force, and isolating a fraction after centrifugation.

The nucleic acid template may comprise an expression template, a translation template, or both an expression template and a translation template, including but not limited to plasmid DNA, linear DNA or mRNA. The template may comprise a construct configured to express the fusion protein. As used herein, the term “construct” refers to a recombinant polynucleotide, i.e., a polynucleotide that was formed artificially by combining at least two polynucleotide components from different sources (natural or synthetic). For example, the constructs described herein comprise a polynucleotide encoding the fusion protein disclosed herein, operably linked to a promoter that (1) is associated with another gene found within the same genome, (2) from the genome of a different species, or (3) is synthetic. Constructs can be generated using conventional recombinant DNA methods. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. The system may comprise one or more polymerases capable of generating a translation template from an expression template.

The terms “nucleic acid,” “nucleic acid sequence,” “polynucleotide,” and “polynucleotide sequence,” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated. The term “promoter” refers to a cis-acting DNA sequence that directs RNA polymerase and other trans-acting transcription factors to initiate RNA transcription from the DNA template that includes the cis-acting DNA sequence.

The term “transcription factor” refers to a protein that regulates transcription of another protein, typically by interacting with one or more cis-acting DNA sequence in or near the promoter for the other protein. A transcription factor may increase expression or decrease expression depending upon whether the transcription factor is activated or deactivated. A transcription factor may become activated or deactivated by an interaction with another molecule (e.g., a target molecule as described above).

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, bacteriophage polymerases such as, but not limited to, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The vector may be a recombinant vector (e.g., a recombinant expression vector) comprising the nucleic acid sequence or construct encoding the fusion protein described herein. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” or “recombinant expression vectors.” One type of vector is a “plasmid”, which refers to a circular double stranded DNA loop into which additional DNA segments may be ligated, specifically exogenous DNA segments encoding the fusion protein.

Cell free reaction mixtures may be incubated with nanocarriers at a specified ratio for 2 hours at room temperature or overnight at 4 °C. An advantage of the method as provided in the present disclosure is the protein of interest can be conjugated to a lipid-based nanocarrier in a short period of time, for example the total time of the method may be less than 10 hrs and not include any living cells.

Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986; 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties).

The fusion protein encoded by the nucleic acid template comprises a functional protein and a second protein that is able to conjugate to the modified lipid. The term “fusion protein” refers to a protein or polypeptide formed from the combination of two different proteins or protein fragments. The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

The second protein may be a protein tag. In exemplary embodiments the protein tag is a self-labeling enzyme. As used herein, a “self-labeling enzyme” are enzyme tags which catalyze the covalent attachment of an exogenously added synthetic ligand. The synthetic ligands are tag specific and can be coupled to diverse useful labels, such as fluorescent dyes, affinity handles, or solid surfaces. Self-labeling enzymes include, without limitation, SNAP-tag, CLIP-tag, HaloTag, and SpyTag. As mentioned above, the modified lipid may be benzylguanine or a BG derivative, and the protein tag may be a SNAP-tag. SNAP-tag is a 182 residues polypeptide (19.4 kDa) that can be fused to any protein of interest and further specifically and covalently tagged with a suitable ligand, such as a fluorescent dye.

In a second aspect, provided herein is a method for functionalizing a nanocarrier is provided, the method comprising incubating the nanoparticle with a cell-free protein synthesis system and a nucleic acid template encoding a fusion protein, the fusion protein comprising a transmembrane domain and a functional protein. The transmembrane domain may be computationally designed to insert into the nanocarrier membrane. A “transmembrane domain” refers to a membrane-spanning protein domain. TMDs may consist of one or several alpha-helices or a transmembrane beta barrel.

In a third aspect, provided herein is a composition comprising a nanocarrier comprising a modified lipid a cell-free protein synthesis system; and a nucleic acid template encoding a fusion protein, the fusion protein comprising a protein tag that conjugates to the modified lipid; and a functional protein. The compositions can be used to prepare functionalized nanocarriers, such as according to the methods described herein. The protein tag may be a self-labeling enzyme, such as a SNAP tag, and the modified lipid may comprise benzylguanine or a BG-derivative.

In a fourth aspect, provided herein is a composition comprising a nanocarrier; a cell-free protein synthesis system; and a nucleic acid template encoding a fusion protein, the fusion protein comprising a transmembrane domain; and a functional protein.

In any of the compositions described herein, the functional protein may be an antibody, an antibody fragment, a de novo designed protein, a receptor, etc. The nanocarrier is a liposome, a polymersome, a lipid nanoparticle, etc. The nanocarrier may further comprise a cargo molecule. A cargo molecule may comprise any molecule which is to be transported or delivered by a lipid-based nanocarrier. By way of example and not limitation lipid-based nanocarrier cargo may comprise RNA, DNA, active pharmaceutical ingredients, adjuvants, proteins, therapeutic cargo, gene editing cargo such as Cas9, delivery of vaccines including mRNA and/or adjuvants. Cargo may be organ, tissue or cell type specific.

As used herein, “active pharmaceutical ingredient” (“API”) or “small molecule drug” refers to a component that provides pharmacological activity or other direct effect in the diagnosis, cure, mitigation, treatment, or prevention of disease, or to affect the structure or any function of the body of man or animals. As used herein, “therapeutic agent” refers to an agent or compound that relieves to some extent one or more signs, symptoms, or causes of a disease or condition.

In a fifth aspect, provided herein is a method of delivering a cargo to a cell, comprising encapsulating the cargo into a lipid-based nanocarrier, functionalizing the nanocarrier according to the methods described herein and dosing the cell with the functionalized nanocarrier, wherein the cell comprises a surface protein that binds the functional protein.

As used herein, the term “encapsulate,” “encapsulated,” or “encapsulation” means enclosed or compartmentalized within a membrane. The membrane provides a semi-permeable barrier between the contents encapsulated within the lumen of the membrane and the external environment. Any methods known in the art for encapsulating cargo may be used.

Dosing the cell may comprise adding the functionalized nanocarrier to the cell at a concentration and/or an amount proportional to the cell at which the intended cargo is delivered. The cell may be an isolated cell in culture, or a cell within an extracted tissue sample, or a cell within an organism in vivo.

The functional protein may allow for specific cell binding, and binding may result in specific cell activation. Upon activation a cell may proliferate, differentiate, migrate or traffic, release proteins or molecules, change morphology or alter cellular function. The cell may be any cell type that would benefit from delivery of the cargo. The cell may comprise a surface protein that recognizes and binds to the functional protein. A lipid-based nanocarrier may comprise a functional protein specific for an immune cell. The functional may be an antigen. The antigen may be an scFv. The immune cell may be a lymphocyte, in particular a T lymphocyte. In an embodiment, the antigen is an scFv specific for a T lymphocyte. Activation of a T lymphocyte includes clonal expansion, differentiation and/or changes in function.

In a sixth aspect, provided herein is a method for generating an artificial antigen presenting cell, the method comprising incubating the nanocarrier with a nucleic acid template encoding a fusion protein and a cell-free protein expression system, wherein the functional protein is an antigen. An antigen presenting cell is a type of immune cell that boosts immune responses by showing antigens on its surface to other cells of the immune system. An artificial antigen presenting cell of the present disclosure may be derived by the methods provided herein, wherein the fusion protein encodes for a binding protein, which can bind to immune cells, in particular lymphocytes. The binding protein may encode for an antigen for which the generation of an immune response is desired. The antigen may be a foreign antigen, for example a pathogen antigen, and endogenous antigen or a tumor antigen. The artificial antigen presenting cell of the present disclosure may conjugate the antigen onto the surface of a lipid-based nanocarrier and bind to immune cells independent of other binding molecules. The fusion protein may encode for a bispecific protein, which can bind to more than one target. When expressed by the antigen presenting cell, the artificial presenting cell itself becomes bispecific. The antigens may include scFvs, such as CD2- and CD3-binding scFv. The artificial antigen presenting cells may be used to activate T lymphocytes.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

The following Examples are illustrative and should not be interpreted to limit the scope of the claimed subject matter.

### Rapid Generation of Therapeutic Nanoparticles Using Cell-Free Expression Systems

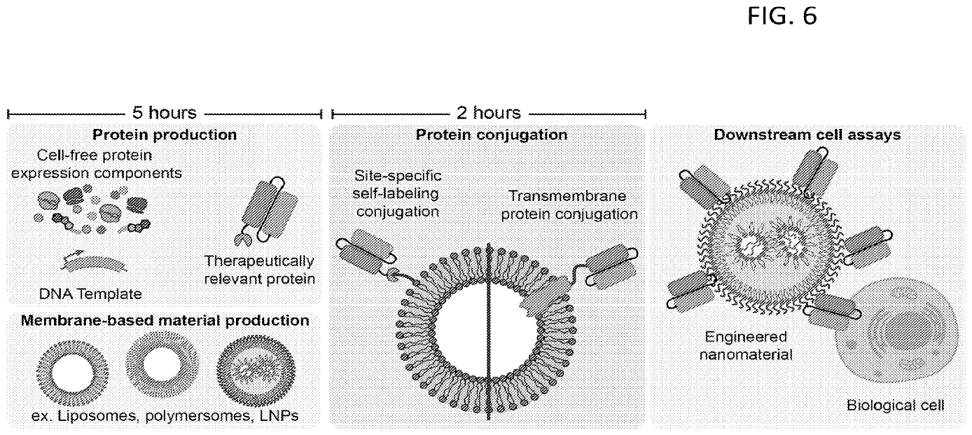

Described herein are methods to rapidly design, express, and conjugate proteins to the surface of membrane-based nanocarriers (FIG. 6). Leveraging rational protein design and cell-free protein synthesis systems, this disclosure provides a generalizable method to rapidly produce protein functionalized therapeutic nanoparticles. Using methods described herein, proteins can be conjugated to nanoparticles in a site-specific manner via chemical conjugation or spontaneously integrated into the carrier membranes via a hydrophobic transmembrane domain. The conjugated proteins not only retain functionality but improve binding specificity due to avidity interactions in vitro. The utility of the platform is further demonstrated by creating bispecific liposome artificial antigen presenting cells (aAPCs) to bind and activate T-cells and lipid nanoparticles (LNPs) to increase functional delivery of mRNA to T-cells.

Rapid expression and conjugation of cell-free expressed proteins to membrane-based materials.

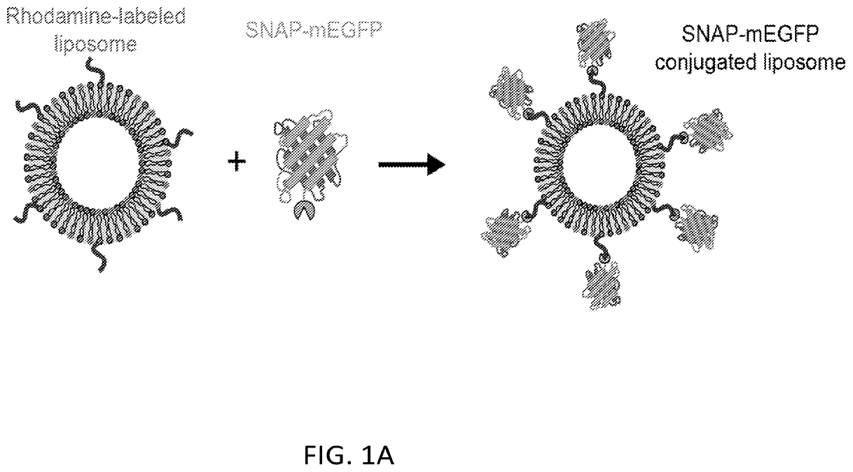

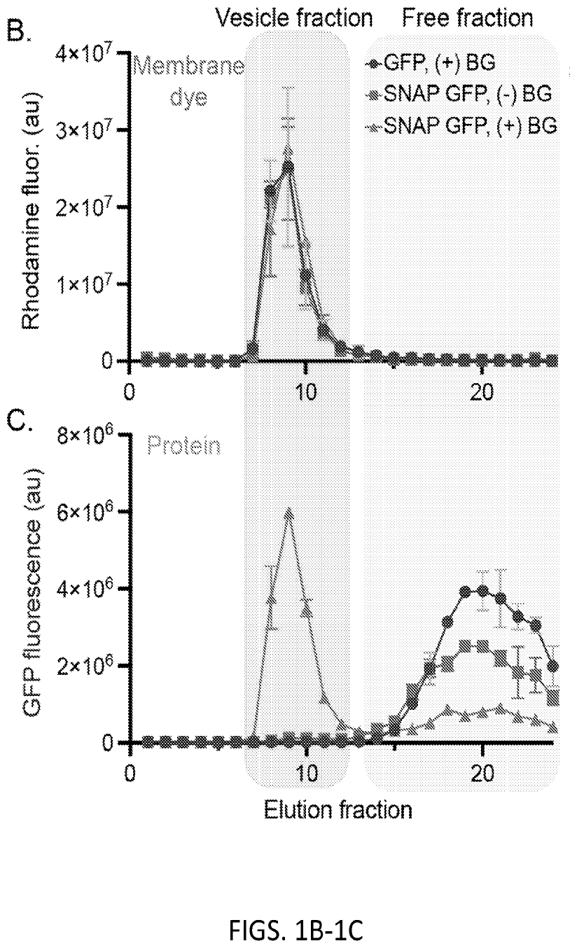

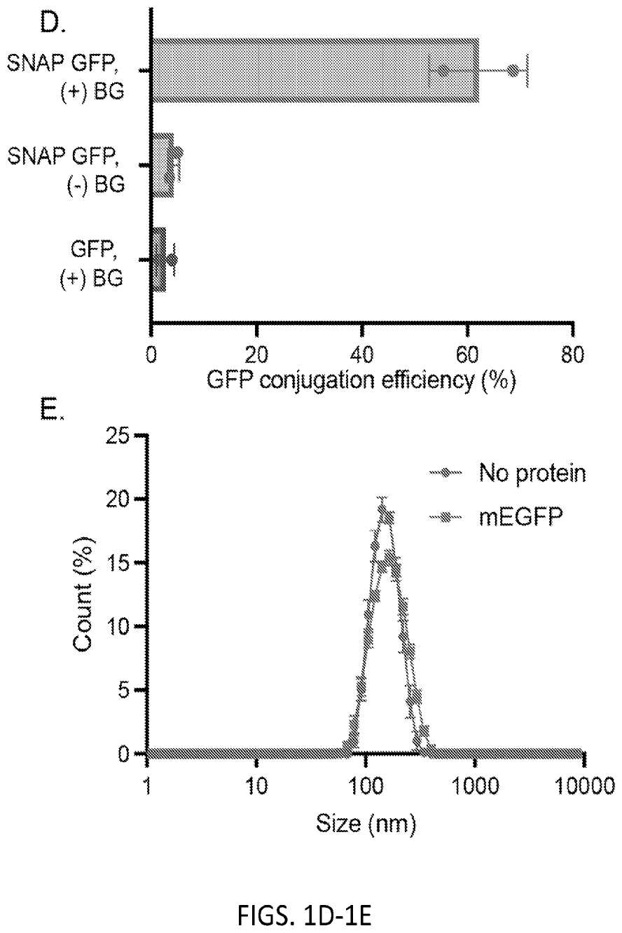

To assess our ability to rapidly produce and conjugate protein to liposomes using a cell-free expression system, we first investigated the expression and conjugation of a model protein, SNAP-monomeric enhanced green fluorescent protein (mEGFP) to liposomes (FIG. 1A). We designed and synthesized a DNA template encoding mEGFP fused to a SNAP tag, a self-labeling enzyme which covalently bonds to benzylguanine, and all the regulatory components required for protein transcription and translation, such as a promoter and ribosome binding site, to allow for direct expression of the protein from a linear template. This enabled us to amplify DNA, without the use of cells, using a polymerase chain reaction. Using this method, we could generate sufficient quantities of purified DNA for cell-free expression encoding our protein of interest in under 2 hours.^{[8]}

To express proteins, we utilized the cell-free system, PURExpress, which contains all the components required for transcription and translation. By combining purified DNA template encoding a protein of interest, with the cell-free expression system, protein can be produced in 3 hours.^{[8]} To enable conjugation of the expressed protein to liposomes, we assembled 100 nm liposomes labeled with lipid conjugated rhodamine dye, with or without 0.1 mol % 18:1 PE PEG2000 benzylguanine.^{[9]} By adding these liposomes into the cell-free reaction mixture, we expected the expressed proteins would be rapidly conjugated to the liposomes and ready for downstream cellular assays in 2 hours (FIG. 6).

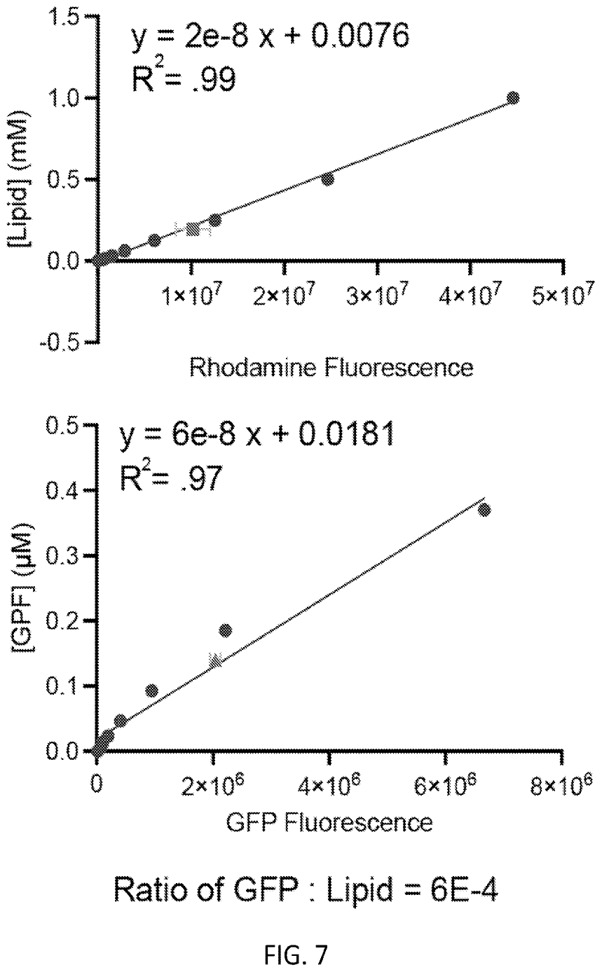

Next, we monitored the efficiency and specificity of protein conjugation to liposomes using size exclusion chromatography. We expressed either SNAP-mEGFP, which should bind the benzylguanine groups on the liposome or mEGFP (-SNAP), which should not bind the liposomes. Both mEGFP and SNAP-mEGFP were incubated with liposomes (+/−benzylguanine) and were then purified using size exclusion chromatography to separate vesicles and vesicle-bound mEGFP from free mEGFP.^{[9]} Elution fraction fluorescence for mEGFP and rhodamine labeled vesicles were then measured to observe the coelution of protein and vesicles for each mixture (FIGS. 1B, C). We found that all vesicles eluted in the same fraction, corresponding to the void volume of the column (FIG. 1B top and vesicle fraction box), but mEGFP fluorescence was only observed in the vesicle fraction when mEGFP was fused to SNAP and mixed with liposomes containing benzylguanine conjugated lipids (FIG. 1B, C). In order to determine the amount of protein conjugated to liposomes, vesicle fractions were collected, and the fluorescence of liposomes and protein were measured and compared to lipid and protein standards. We determined that we achieved about 60% conjugation efficiency (or 6e-4 mol protein to 1 mol lipid) based on the added 0.1 mol % 18:1 PE PEG2000 benzylguanine lipid added to our membranes (FIGS. 1D and 7). Interestingly, this efficiency corresponds to about the theoretical maximum conjugation efficiency of SNAP protein to benzylguanine lipids, as slightly less than half of the benzylguanine lipid should be on the inner leaflet of liposomes, where it is inaccessible to protein. Lastly, we confirmed that liposomes conjugated with protein retained their size via dynamic light scattering measurements. Vesicles with and without protein were found to be approximately the same size, with a diameter of ~140 nm (FIG. 1E). Combined, these data demonstrate that we can rapidly produce and conjugate cell-free expressed proteins to liposomes in 7 hours.

Cell-free expressed proteins enable specific binding of lipid-based nanoparticles to cells.

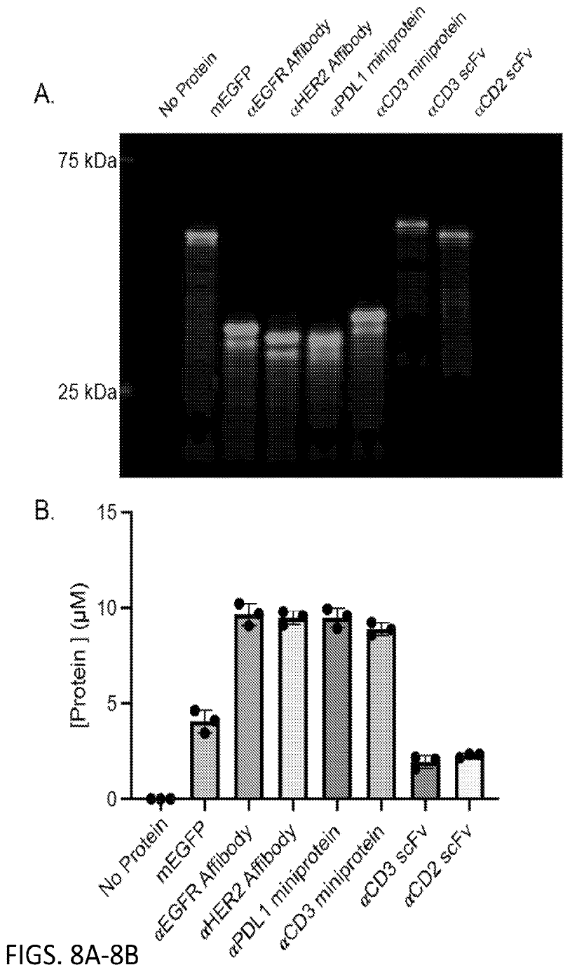

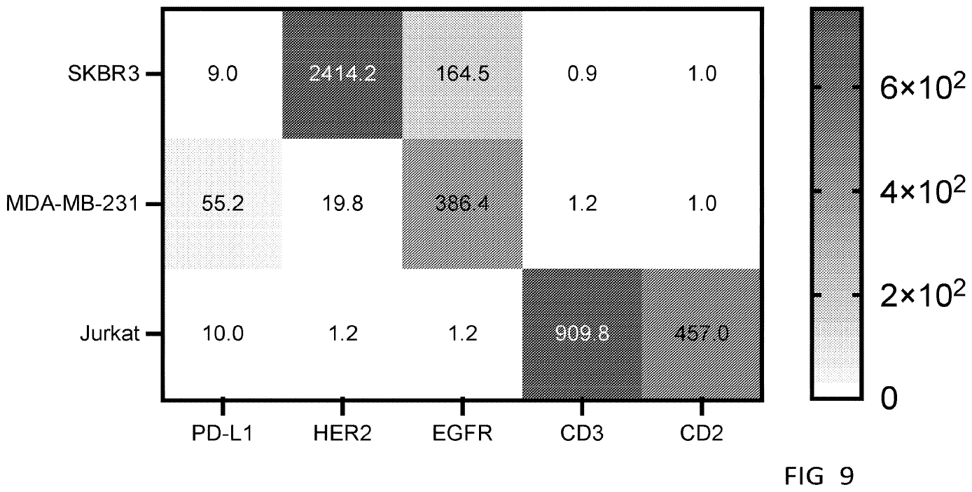

Once we established that we could conjugate cell-free expressed mEGFP to the surface of liposomes, we tested our ability to express and conjugate functional binding proteins. We selected a panel of structurally diverse binding proteins, including affibodies, computationally designed miniproteins, and scFvs which bind therapeutically relevant proteins including HER2,^{[10]} EGFR,^{[11]} PDL1,^{[12]} CD3,^{[13]} and CD2^{[14]}. Protein expression was confirmed and quantified via SDS-PAGE (FIG. 8). Functional binding to cells was then tested for either soluble or vesicle bound proteins via flow cytometry. To assess soluble protein binding, proteins were labeled with SNAP547 dye, while vesicle-bound protein binding was detected by the presence of a rhodamine-conjugated lipid present in liposome membranes. We assessed protein binding to three cell types: Jurkat, an immortalized T-cell line which expresses CD2 and CD3, MDA-MB-231, an immortalized breast cancer cell line which expresses EGFR, and SKBR3, an immortalized HER2/EGFR positive breast cancer cell line. Each cell-type differentially expresses our protein targets of interest, allowing for the specificity of binding by each protein or protein-liposome complex to be characterized (FIG. 9).

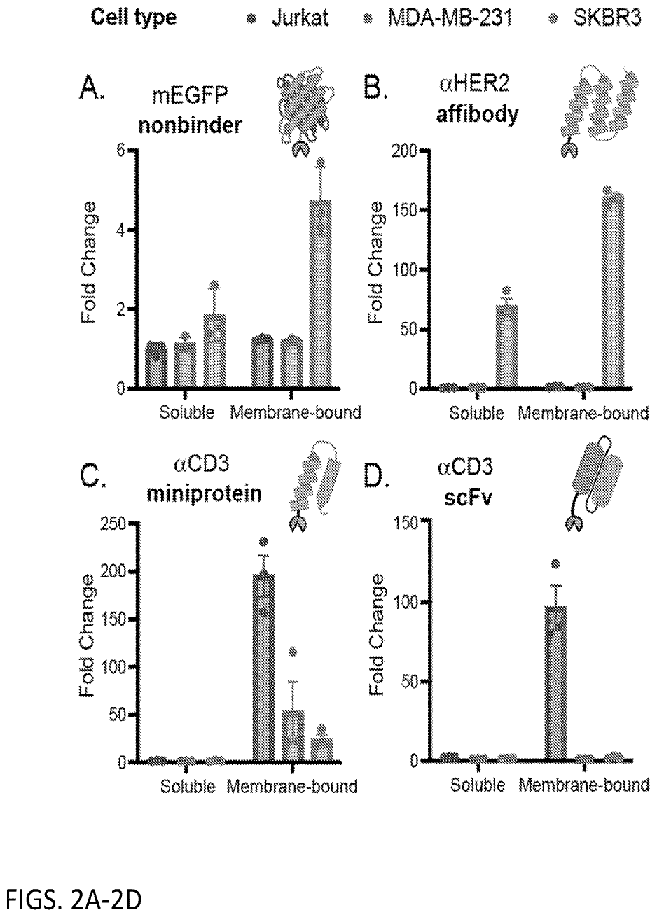

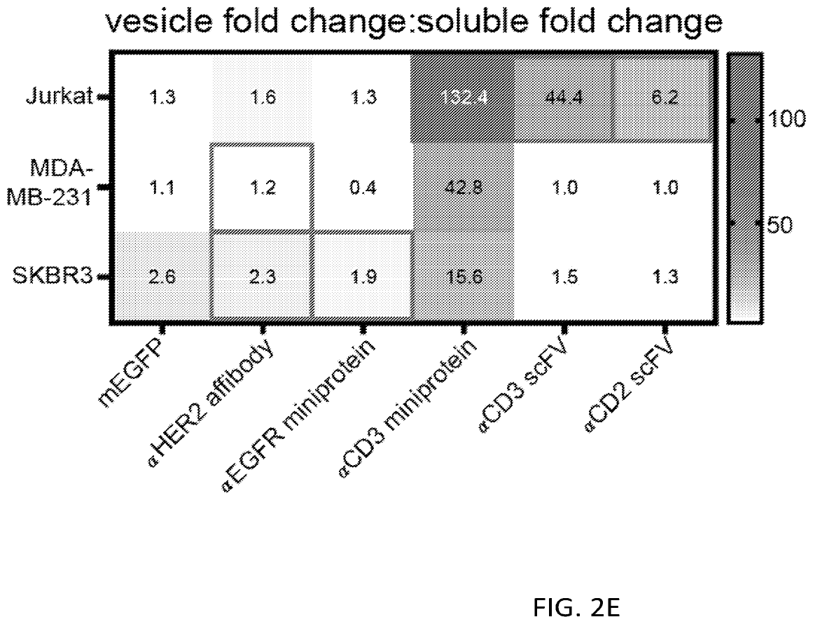

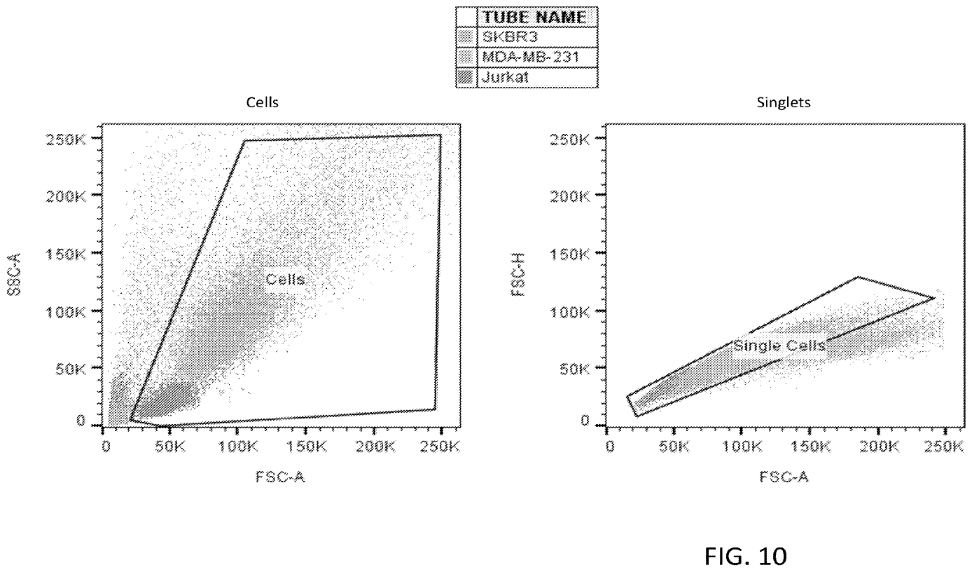

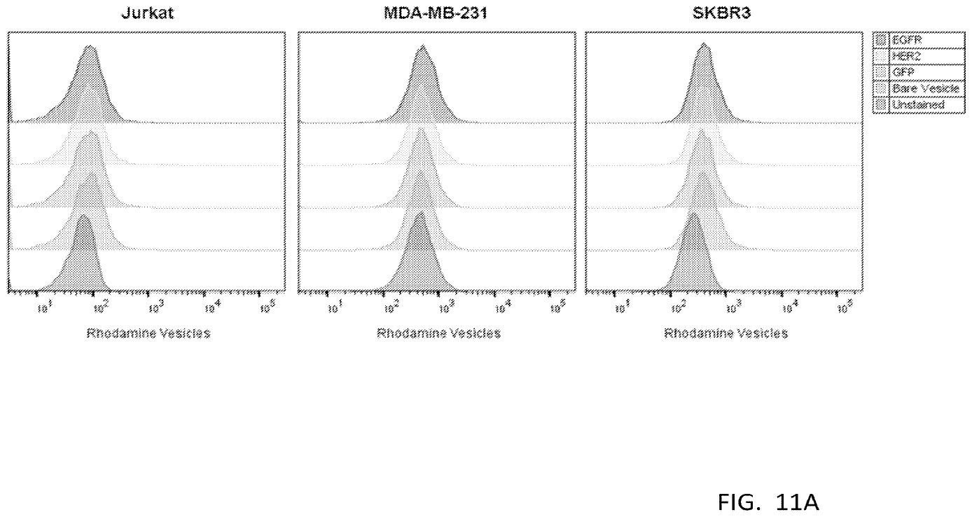

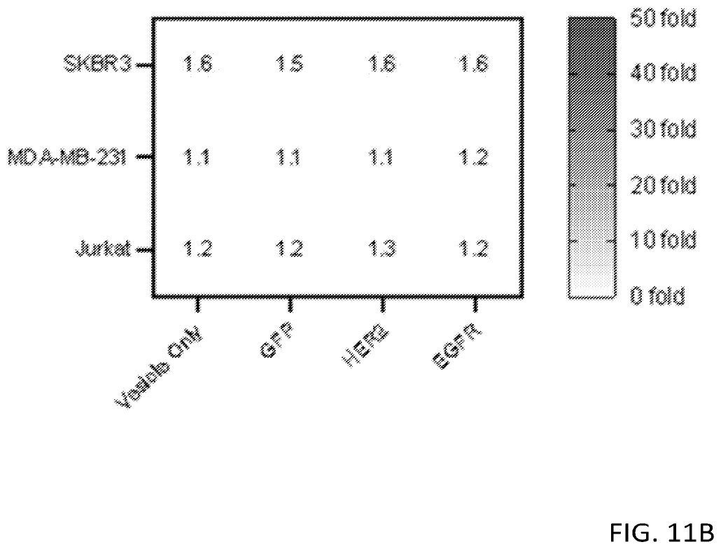

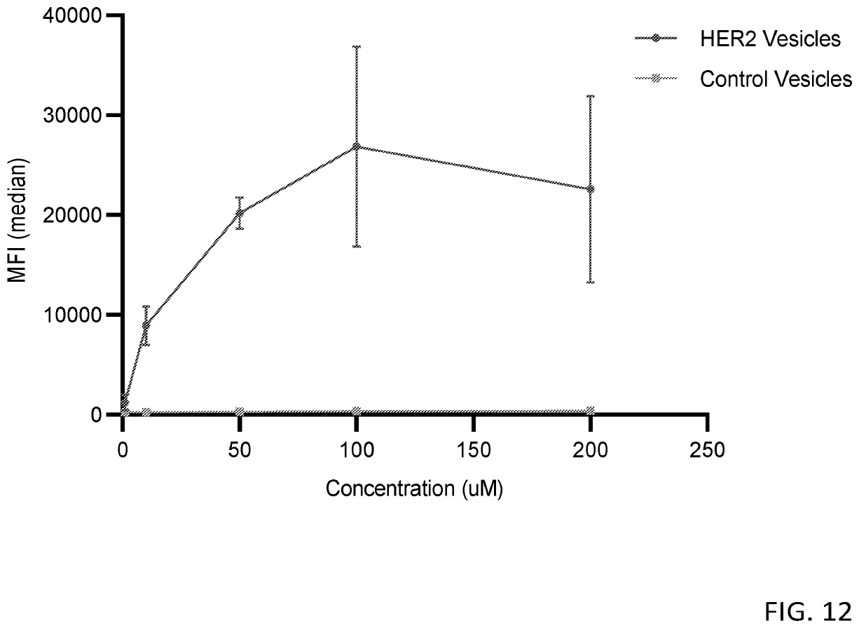

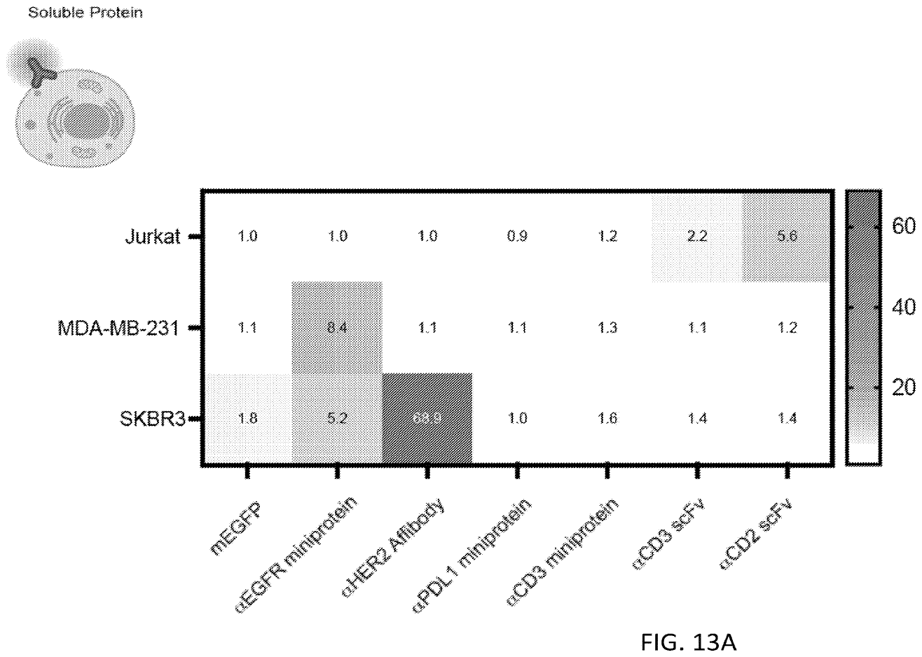

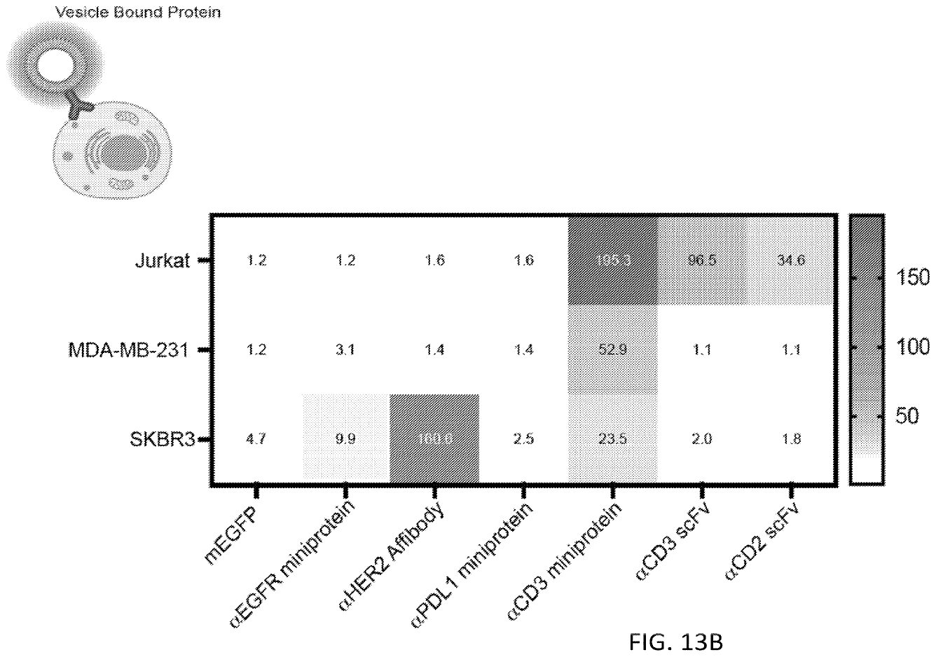

We characterized the binding of expressed protein to cells as a function of their conjugation-state—either soluble or conjugated to liposomes—via flow cytometry (FIG. 10). We first established that liposomes did not bind cells when proteins were not conjugated to the liposome surface by incubating cells with either the binding proteins or liposomes without benzylguanine lipid (FIG. 11). As a negative control, we then measured mEGFP binding to each cell type. Interestingly, mEGFP was found to have a slight affinity to SKBR3 cells, but overall, very weakly bound to cells both in soluble and liposome conjugated forms as expected (FIG. 2A). We found that the αHER2 affibody, αEGFR miniprotein, and the αCD3 and αCD2 scFvs bound the correct cells when in soluble or vesicle bound form (FIGS. 2B and D, FIGS. 12, 13).^{[10,13-15]} The αPDL1 miniprotein appeared to express as determined by SDS-PAGE, however no cellular binding was detected, suggesting that the protein was likely improperly folded. We hypothesize that improper folding occurred due to the presence of 3 disulfide bonds and could potentially be remediated by optimizing cell-free reaction conditions to better support disulfide bond formation.^{[12,16]} Notably, the computationally designed αCD3 miniprotein bound weakly to all cells when in a soluble form, however it bound more strongly to cells when conjugated to liposomes. Binding for the miniprotein was found to be somewhat non-specific: while the αCD3 miniprotein strongly bound Jurkat cells, which have surface expressed CD3, it also mildly bound to the other cell-lines which do not express CD3 on their surfaces (FIG. 2C). When assessing αCD3 miniprotein-conjugated liposome size via DLS, we observed an increase in vesicle diameter, suggesting that miniproteins may oligomerize, causing vesicle aggregation and subsequent nonspecific binding to cells (Table 1). Interestingly, we found that proteins often bound more strongly to cells when conjugated to liposomes, likely due to avidity effects (FIG. 2E). Notably, this phenomena enabled the αCD3 scFv to significantly increase binding to target cells, but also increased nonspecific binding in the case of mEGFP to SKBR3 cells. Together, these data demonstrate that liposomes can be functionalized with structurally diverse, active, and therapeutically relevant cell-free expressed proteins.

TABLE 1
Particle size and polydispersity for
nanocarriers produced in this study.

| | Peak Intensity | | Polydispersity |
|---|---|---|---|
| Nanocarrier | Mean (SD) | | Index (SD) |
| BG Vesicles | 154 | (13) | 0.08 (0.03) |
| anti-HER2 BG vesicles | 172 | (9) | 0.20 (0.02) |
| anti-EGFR BG vesicles | 181 | (7) | 0.18 (0.03) |
| anti-GFP BG vesicles | 171 | (7) | 0.13 (0.01) |
| anti-CD3 miniprotein BG | 270 | (30) | 0.42 (0.06) |
| vesicles | | | |
| anti-CD3 scFV vesicles | 150 | (5) | 0.17 (0.02) |
| anti-CD2 BG vesicles | 157 | (4) | 0.17 (0.01) |
| LNP | 164 | (5) | 0.14 (0.01) |
| LNP-GFP | 170 | (4) | 0.17 (0.01) |
| LNP-HER2 | 167 | (3) | 0.17 (0.01) |
| Polymer | 102 | (4) | 0.18 (0.02) |
| Polymer GFP | 128 | (37) | 0.29 (0.04) |
| Polymer HER2 | 99 | (6) | 0.24 (0.02) |
| Ves ctrl | 113 | (13) | 0.11 (0.04) |
| TMD - GFP | 114 | (10) | 0.10 (0.02) |
| TMD - HER2 | 111 | (5) | 0.11 (0.02) |

Cell-free expressed protein conjugation is indiscriminate to conjugation method and particle type.

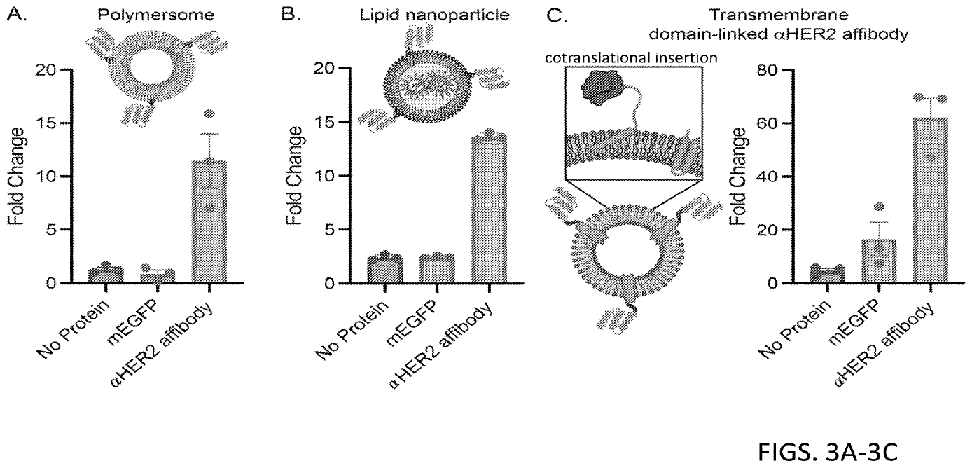

To explore the generalizability of this method, we then tested our ability to conjugate proteins to different types of membrane-based nanoparticles and alter the membrane conjugation strategy. Beyond liposomes, there exist a wider range of membrane-based nanoparticles that might be advantageous to use for a given application. This is because different types of nanoparticles possess different properties which may affect drug availability and pharmacokinetics. [17,18] To demonstrate our results conjugating cell-free expressed proteins to liposomes is generalizable to other nanoparticles, we conjugated proteins to polymersomes and lipid-nanoparticles. We made polymersomes from the diblock copolymer, poly(ethylene oxide)₉-b-poly butadiene₁₂ (1.05 kDa PEO-b-PBD) and lipid-nanoparticles with 0.1 mol % PE PEG2000 benzylguanine conjugated lipid and functionalized them with cell-free expressed αHER2 affibody. Following conjugation, we assessed each particle type's ability to bind to SKBR3 cells via flow cytometry. We found that all particle types could be conjugated to cell-free expressed protein and bind cells (FIGS. 3A, B). The ability to modify different compositions of nanoparticles with cell-free expressed proteins could enable the design of protein-conjugated nanoparticles with diverse properties.

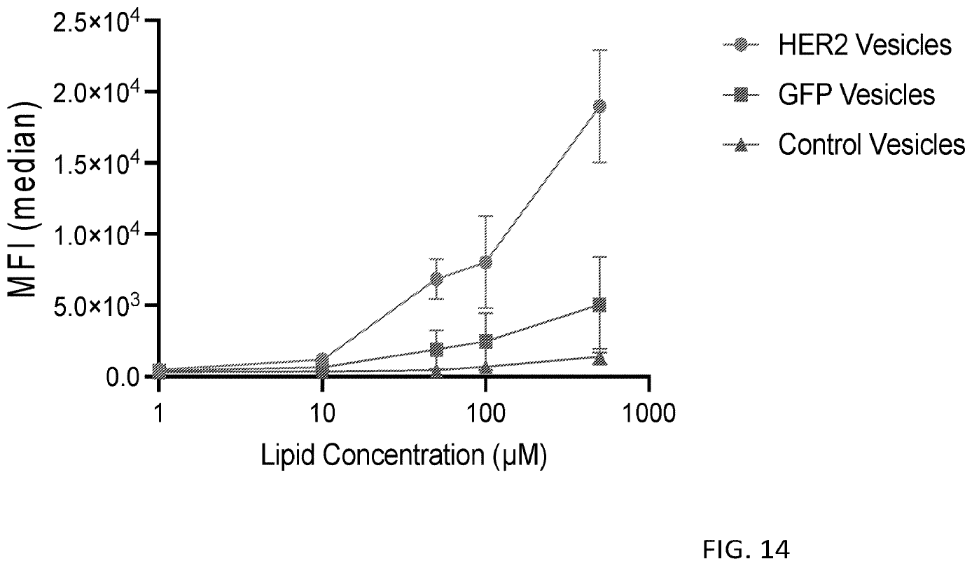

Until this point, our vesicle conjugation approach was to attach soluble proteins to vesicles via a self-labeling enzyme (SNAP tag). Because many therapeutically relevant proteins, especially those used for targeting purposes, are membrane proteins containing transmembrane domains, we sought to confirm that proteins of this type could also be conjugated to the membrane-based materials in our study. Briefly, we measured the capacity of vesicles containing proteins that were co-translationally integrated into vesicle membranes to bind target cells. This integration mode mimics that of cells, as it utilizes a transmembrane domain inserted into the lipid bilayer to anchor or display a protein of interest to the nanoparticle surface. This conjugation strategy only relies on simple hydrophobic interactions and does not require complex membrane chemistries. To accomplish this design, we fused a computationally designed transmembrane domain to the αHER2 affibody.^{[19,20]} By including liposomes in the cell-free reaction, cell-free expressed transmembrane domains could co-translationally insert into liposome membranes, thus decorating particles with αHER2 affibody.^{[20]} We then assessed liposome binding to HER2 expressing SKBR3 cells when decorated with no protein, transmembrane fused mEGFP, and transmembrane fused αHER2 affibody. We found that liposomes with transmembrane fused αHER2 affibody strongly bound to cells (FIG. 3C, FIG. 14). Liposomes without protein did not measurably bind to cells, however transmembrane fused mEGFP liposomes did exhibit non-specific binding. From FIG. 2A, it appears that SKBR3 cells have a slight affinity for mEGFP compared to other cell types. This effect is likely enhanced in the case of transmembrane fused mEGFP as the liposomes used did not possess PEGylated lipids commonly used to reduce nonspecific binding of nanoparticles to cells. Together, these data demonstrate that our method is applicable to multiple particle and protein types as well as conjugation strategies.

Cell-free expressed proteins can interact with, activate, and deliver cargo to T-cells.

Once we established that we could functionalize membrane-based materials with functional cell-free expressed binding proteins, we explored the capacity of these particles to act as therapeutically-relevant nanoparticles. Specifically, we assessed the extent to which protein-modified nanoparticles could act as aAPCs to activate T-cells as well as nucleic acid delivery vehicles to transfect T-cells.

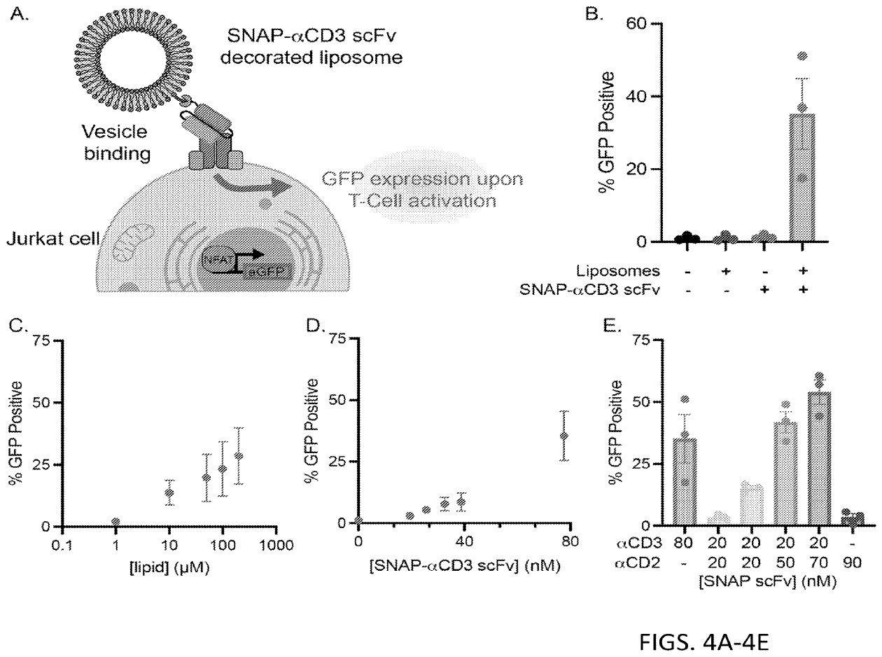

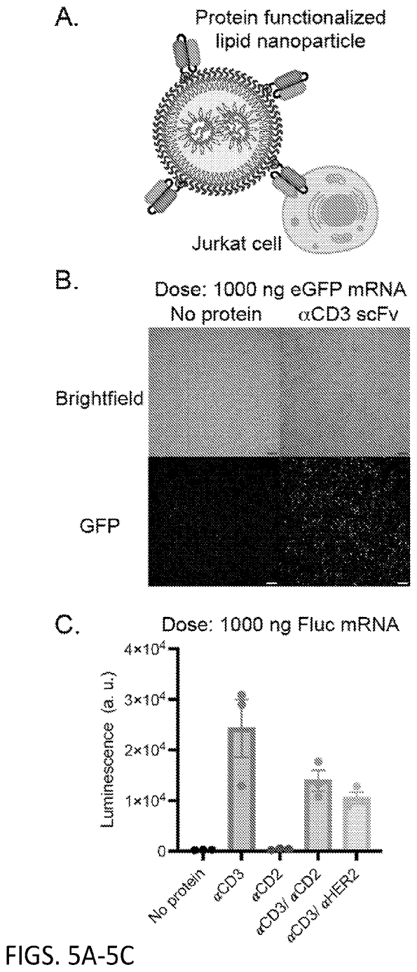

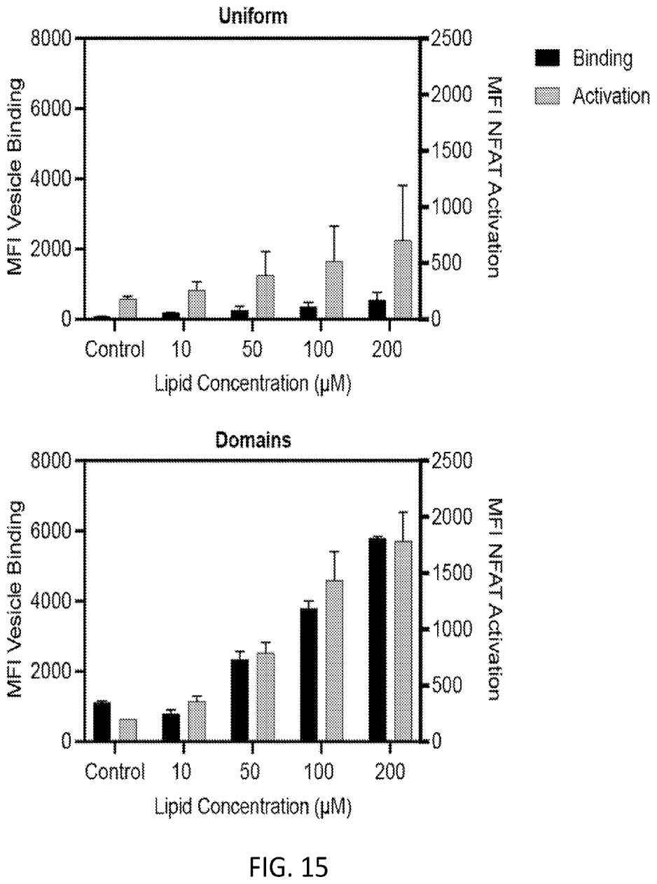

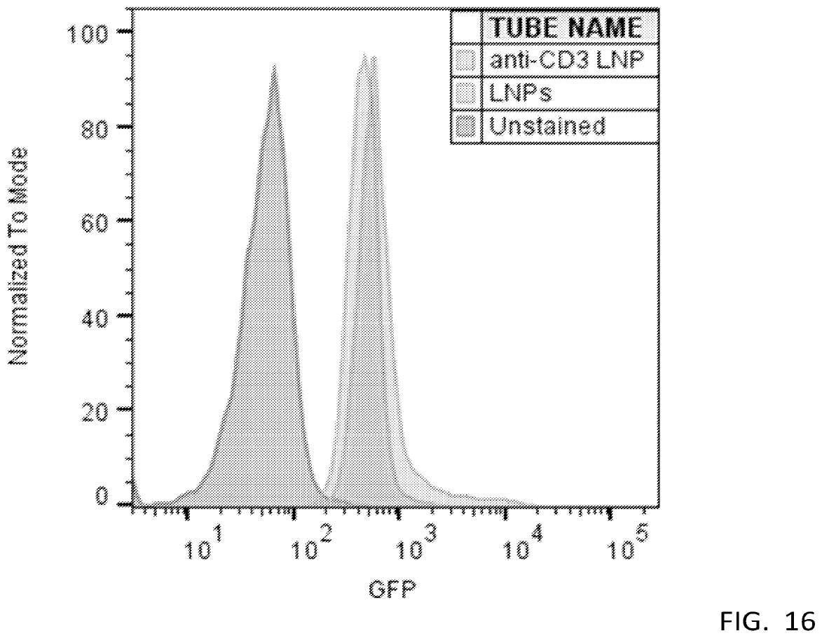

To produce aAPCs, we expressed and conjugated αCD3 scFv to liposomes. To assess the ability of synthetic aAPCs to interact with and activate T-cells, we incubated nanoparticles with a Jurkat reporter cell line that expresses eGFP upon activation and assessed cell activation via flow cytometry (FIG. 4A). This reporter cell line utilizes the nuclear transcription factor NFAT (Nuclear Factor of Activated T cells), which activates after T cell receptor/CD3 engagement and calcium influx to induce gene expression of eGFP. We found that T-cells expressed eGFP when incubated with αCD3 scFv conjugated liposomes, but not when incubated with protein-free liposomes or soluble protein (FIG. 4B). This result agrees with previous work that αCD3 scFv in a soluble, non-liposomal form cannot activate T cells alone because scFv cannot induce TCR dimerization, while our αCD3 aAPCs induces TCR multimerization.^{[21,22]} Once we established that αCD3 scFv conjugated aAPCs activated T-cells, we characterized how lipid dose and protein coverage affected activation. We found that T-cell activation was sensitive to both lipid dose and protein coverage, as GFP expression increased with lipid and protein concentration (FIG. 4C, D). We then explored how protein spatial presentation, through the introduction of lipid domains in liposome membranes, affected binding and activation of Jurkats. Lipid domains, or compartmentalized membrane regions with distinct lipid compositions, have been shown to affect liposome binding and interaction with cells.^{[23]} By doping in saturated DSPC (18:0 PC) into our liposome membranes, lipids spontaneously phase separate causing the benzylguanine lipid, and the bound αCD3 protein, to cluster. We found that phase-separated αCD3 aAPCs bound and activated T-cells more strongly compared to homogenous αCD3 domain aAPCs (FIG. 15). Lastly, we explored our ability to enhance T-cell activation by conjugating both αCD3 and αCD2 scFv to liposomes to produce bispecific aAPCs. CD2 is a costimulatory receptor that is known to vastly amplify T cell signaling.^{[24,25]} While αCD2 antibodies have been found on aAPCs in combination with other costimulatory antibodies like αCD28, they are rarely used as the sole costimulatory signal.^{[26,27]} We reduced the amount of αCD3 scFv on liposomes down to a regime which did not activate T-cells (20 nM). We then maintained this lower amount of αCD3 but varied the amount of αCD2 scFv conjugated to liposomes. We found that by increasing the amount of αCD2 scFv on liposomes, T-cell activation could be enhanced above the level of activation of T-cells when conjugated αCD3 scFv alone. T-cells did not activate when incubated with liposomes conjugated only to αCD2 scFv (FIG. 4E). The modification of nanoparticles with cell-free expressed protein provides a platform to assemble and engineering liposome-based bispecifics and the rapid exploration of costimulatory effects of multiple ligands on nanoparticle surfaces.

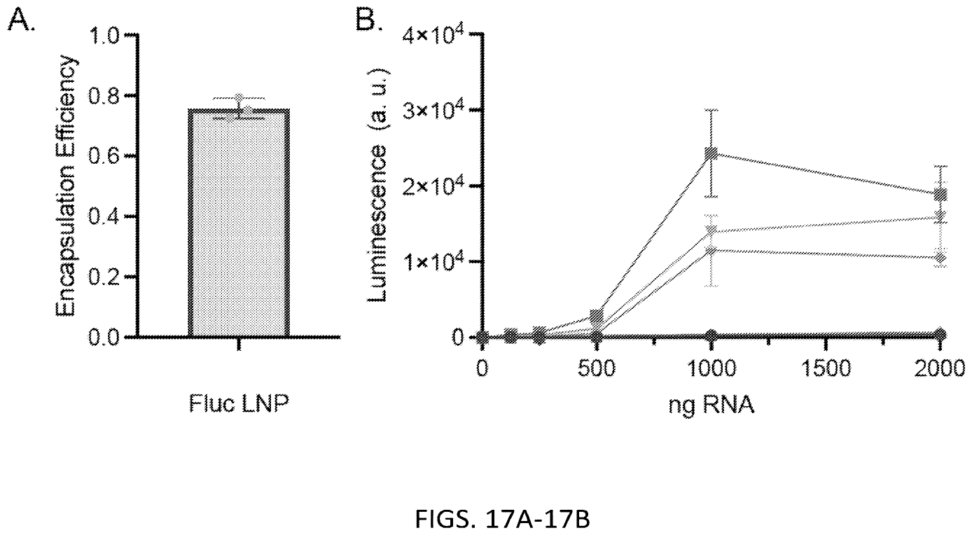

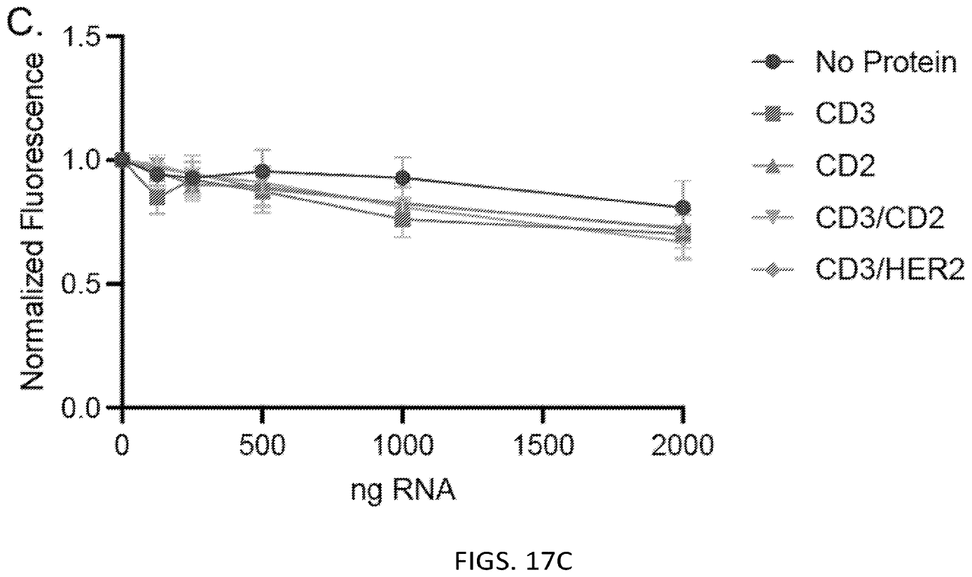

We then asked if we could leverage these proteins to selectively transfect T cells. T cells are notoriously difficult to transfect. The ability to reprogram T cells in vivo could enable the creation of cancer fighting CAR-T cells. It has previously been shown that αCD3-functionalized LNPs can deliver mRNA to T-cells, however in that study, recombinantly expressed proteins were used.^{[28]} To test the utility of our system, we sought to recreate these studies with our cell-free expressed αCD3 scFv. We first tested LNPs functionalized with cell-free expressed αCD3 scFv to functionally deliver mRNA to T cells (FIG. 5A). We encapsulated mRNA encoding eGFP into LNPs and conjugated either no protein or αCD3 scFv to their surface. We observed much stronger GFP expression when T cells were dosed with αCD3 scFv-conjugated LNPs compared to protein-free particles as assessed by microscopy and flow cytometry (FIG. 5B, FIG. 16). Once we established that αCD3 scFv functionalized LNPs enabled the functional delivery of mRNA to T cells, we wondered how delivery could be altered by conjugating multiple proteins to the surface of nanoparticles. We assembled LNPs encapsulating mRNA encoding firefly luciferase (Fluc) and functionalized the nanoparticle surfaces with αCD3 scFv, αCD2 scFv, αCD3 scFv and αCD2 scFv, or αCD3 scFv and αHER2 affibody (a nonbinder) and assessed their ability to functionally deliver mRNA to Jurkat cells (FIG. 5C, FIG. 17). LNPs decorated with αCD3 scFv exhibited the highest luciferase expression, followed by LNPs conjugated to αCD3 scFv and αCD2 scFv and αCD3 scFv and αHER2 affibody (nonbinder). Minimal luminescence was detected when Jurkat cells were dosed with protein-free LNPs or LNPs conjugated to αCD2 scFv only (FIG. 5B, FIG. 17). Combined, these studies demonstrate that therapeutically relevant, protein-decorated nanoparticles can be generated in a matter of hours using cell-free expression systems.

Here, we explored how cell-free protein expression systems could be used to rapidly generate therapeutically relevant membrane-based materials. We demonstrated that we could quickly express and conjugate cell-free expressed binding proteins to liposomes, polymersomes, and lipid nanoparticles. We then demonstrated that this method enables the production of bispecific artificial antigen presenting cells and enhances lipid nanoparticle-mediated delivery of mRNA to T cells.

This work demonstrates the utility of cell-free systems for the rapid development of protein-functionalized membrane-based materials. We showed that cell-free expression systems can be used to produce multiple types of binding proteins, including affibodies, de novo designed proteins, and scFvs. These proteins can be decorated onto multiple type of particles, including liposomes, polymersomes, and lipid nanoparticles, via a self-labeling enzyme or cell-mimetic transmembrane domain. Using this strategy, we demonstrated the rapid development of a bispecific artificial antigen presenting cell and enhancement in delivery of mRNA to T-cells. We believe that this workflow can bolster our ability to design, build, and test cell-mimetic nanocarriers, ultimately fast tracking the delivery of more effective therapeutics to the clinic.

### Experimental Section

### Materials

Lipids 1,2-dioleoyl-sn-glycero-3-phosphocholine (DOPC), 1,2-distearoyl-sn-glycero-3-phosphocholine (DSPC), cholesterol, 1,2-dioleoyl-sn-glycero-3-phosphoethanolamine-N-[benzylguanine (polyethylene glycol)-2000] (BG), 1,2-distearoyl-sn-glycero-3-phosphoethanolamine-N-[methoxy (polyethylene glycol)-2000] (ammonium salt), 1,2-dioleoyl-sn-glycero-3-phosphoethanolamine-N-(lissamine rhodamine B sulfonyl) (ammonium salt), 1,2-dioleoyl-sn-glycero-3-phosphoethanolamine-N-(Cyanine 5.5) were purchased from Avanti Polar Lipids. D-Lin-MC3-DMA lipid was purchased from Fisher Scientific. NFAT eGFP Reporter Lentivirus was purchased from BPS Biosciences. Antibodies mouse IgG1 isotype (MOPC-21), PE anti-human CD3 (UCHT1), PE anti-human CD2 (RPA-2.10), PE anti-human PD-L1 (MIH2), PE anti-human CD340 (erbB2/HER-2, 24D2), and PE anti-human EGFR (AY13) were purchased from Biolegend.

### Methods

### DNA Design and Preparation

All genes were ordered as linear templates from Twist Biosciences. DNA templates were designed with a T7 promoter and terminator, as well as a ribosome binding site (Table 2) and were amplified via PCR and purified using a PureLink PCR Purification Kit (Invitrogen). PCR products were used directly in cell-free reactions.

TABLE 2
DNA sequences used in this study.

| Protein and | SEQ | |
|---|---|---|
| Source | ID NO: | DNA Sequence |
| SNAP-mEGFP | 1 | TAATACGACTCACTATAGGGAGACCACAACGGTTTCCC |
| [20] | | TCTAGAAATAATTTTGTTTAACTTTAAGAAGGAGATATA |
| | | CATATGGACTATAAAGACGATGACGATAAACACATGGG |
| | | TAGCATGGACAAAGATTGCGAAATGAAACGTACCACCC |
| | | TGGATAGCCCGCTGGGCAAACTGGAACTGAGCGGCTGC |
| | | GAACAGGGCCTGCATGAAATTAAACTGCTGGGTAAAGG |
| | | CACCAGCGCGGCCGATGCGGTTGAAGTTCCGGCCCCGG |
| | | CCGCCGTGCTGGGTGGTCCGGAACCGCTGATGCAGGCG |
| | | ACCGCGTGGCTGAACGCGTATTTTCATCAGCCGGAAGC |
| | | GATTGAAGAATTTCCGGTTCCGGCGCTGCATCATCCGGT |
| | | GTTTCAGCAGGAGAGCTTTACCCGTCAGGTGCTGTGGA |
| | | AACTGCTGAAAGTGGTTAAATTTGGCGAAGTGATTAGC |
| | | TATCAGCAGCTGGCGGCCCTGGCGGGTAATCCGGCGGC |
| | | CACCGCCGCCGTTAAAACCGCGCTGAGCGGTAACCCGG |
| | | TGCCGATTCTGATTCCGTGCCATCGTGTGGTTAGCTCTA |
| | | GCGGTGCGGTTGGCGGTTATGAAGGTGGTCTGGCGGTG |
| | | AAAGAGTGGCTGCTGGCCCATGAAGGTCATCGTCTGGG |
| | | TAAACCGGGTCTGGGAGGTAGCTCAGGAGCAAGTCCGG |
| | | CAGCACCGGCACCGGCATCACCAGCAGCTCCAGCACCT |
| | | AGTGCACCGGCAGGCGGTATGGTTAGCAAAGGTGAAGA |
| | | ACTGTTTACCGGTGTTGTTCCGATTCTGGTTGAACTGGA |
| | | TGGTGATGTTAATGGCCACAAATTTTCAGTTAGCGGTGA |
| | | AGGCGAAGGTGATGCAACCTATGGTAAACTGACCCTGA |
| | | AATTTATCTGTACCACCGGCAAACTGCCGGTTCCGTGGC |
| | | CGACACTGGTTACCACACTGACCTATGGTGTTCAGTGTT |
| | | TTAGCCGTTATCCGGATCATATGAAACAGCACGATTTTT |
| | | TCAAAAGCGCAATGCCGGAAGGTTATGTTCAAGAACGT |
| | | ACCATCTTCTTCAAAGATGACGGCAACTATAAAACCCG |
| | | TGCCGAAGTTAAATTTGAAGGTGATACCCTGGTGAATC |
| | | GCATTGAACTGAAAGGCATCGATTTTAAAGAGGATGGT |
| | | AATATCCTGGGCCACAAACTGGAATATAATTATAATAG |
| | | CCACAACGTGTACATCATGGCCGACAAACAGAAAAATG |
| | | GCATCAAAGTGAACTTCAAGATCCGCCATAATATTGAA |
| | | GATGGTTCAGTTCAGCTGGCAGATCACTATCAGCAGAA |
| | | TACCCCGATTGGTGATGGTCCGGTTCTGCTGCCGGATAA |
| | | TCATTATCTGAGCACCCAGAGCAAACTGAGCAAAGATC |
| | | CGAATGAAAAACGTGATCACATGGTGCTGCTGGAATTT |
| | | GTTACCGCAGCAGGTATTACCTTAGGTATGGATGAACT |
| | | GTACAAATAAGTCGACCGGCTGCTAACAAAGCCCGAAA |
| | | GGAAGCTGAGTTGGCTGCTGCCACCGCTGAGCAATAAC |
| | | TAGCATAACCCCTTGGGGCCTCTAAACGGGTCTTGAGG |
| | | GGTTTTTTG |
| Transmembrane | 2 | TAATACGACTCACTATAGGGGAATTGTGAGCGGATAAC |
| mEGFP | | AATTCCCCTCTAGAAATAATTTTGTTTAACTTTAAGAAG |
| [20] | | GAGATATACCATGGGTAGCACCCGTACCGAAATTATCA |
| | | CCCGTCTGAGCTTTAGCCTGCTGCTGCAGCTGGTTCTGG |
| | | CAATTTTTCTGCTGGCACTGCTGATTGTTCTGCTGTGGC |
| | | TGCTGCAACAGCTGAAAGAACTGCTGCGTGAACTGGAA |
| | | CGTCTGCAGCGTGAAGGTAGCAGTGATGAAGATGTTCG |
| | | TGAGCTGCTGCGCGAAATTAAAGAACTGGTTGAAAACA |
| | | TTGTGTATCTGGTGATCATTATCATGGTTCTGGTGCTGG |
| | | TTATTATTGCACTGGCAGTTCTGCAGATGTACCTGGTTC |
| | | GGGAACTGAAACGTCAGGATGGTAGCAGCGGTAGCATG |
| | | GTTAGCAAAGGTGAAGAACTGTTTACCGGTGTTGTTCC |
| | | GATTCTGGTTGAACTGGATGGTGATGTTAATGGCCACA |
| | | AATTTTCAGTTAGCGGTGAAGGCGAAGGTGATGCAACC |
| | | TATGGTAAACTGACCCTGAAATTTATCTGTACCACCGGC |
| | | AAACTGCCGGTTCCGTGGCCGACACTGGTTACCACACT |
| | | GACCTATGGTGTTCAGTGTTTTAGCCGTTATCCGGATCA |
| | | TATGAAACAGCACGATTTTTTCAAAAGCGCAATGCCGG |
| | | AAGGTTATGTTCAAGAACGTACCATCTTCTTCAAAGATG |
| | | ACGGCAACTATAAAACCCGTGCCGAAGTTAAATTTGAA |
| | | GGTGATACCCTGGTGAATCGCATTGAACTGAAAGGCAT |
| | | CGATTTTAAAGAGGATGGTAATATCCTGGGCCACAAAC |
| | | TGGAATATAATTATAATAGCCACAACGTGTACATCATG |
| | | GCCGACAAACAGAAAAATGGCATCAAAGTGAACTTCAA |
| | | GATCCGCCATAATATTGAAGATGGTTCAGTTCAGCTGG |
| | | CAGATCACTATCAGCAGAATACCCCGATTGGTGATGGT |
| | | CCGGTTCTGCTGCCGGATAATCATTATCTGAGCACCCAG |
| | | AGCAAACTGAGCAAAGATCCGAATGAAAAACGTGATC |
| | | ACATGGTGCTGCTGGAATTTGTTACCGCAGCAGGTATTA |
| | | CCTTAGGTATGGATGAACTGTACAAATAAGGATCCGGC |
| | | TGCTAACAAAGCCCGAAAGGAAGCTGAGTTGGCTGCTG |
| | | CCACCGCTGAGCAATAACTAGCATAACCCCTTGGGGCC |
| | | TCTAAACGGGTCTTGAGGGGTTTTTTG |
| SNAP-αHER2 | 3 | TAATACGACTCACTATAGGGAGACCACAACGGTTTCCC |
| affibody | | TCTAGAAATAATTTTGTTTAACTTTAAGAAGGAGATATA |
| [10] | | CATATGGACTATAAAGACGATGACGATAAACACATGGG |
| | | CAGCATGGATAAAGATTGCGAAATGAAACGTACCACAC |
| | | TGGATAGTCCGCTGGGTAAACTGGAACTGAGCGGTTGT |
| | | GAACAGGGTCTGCATGAAATTAAACTGTTAGGTAAAGG |
| | | CACCAGCGCAGCAGATGCAGTTGAAGTTCCGGCACCGG |
| | | CAGCAGTTTTAGGTGGTCCGGAACCGCTGATGCAGGCA |
| | | ACCGCATGGCTGAATGCATATTTTCATCAGCCGGAAGC |
| | | CATTGAAGAATTTCCGGTTCCTGCACTGCATCATCCGGT |
| | | TTTTCAGCAAGAAAGCTTTACCCGTCAGGTTCTGTGGAA |
| | | ACTGCTGAAAGTTGTTAAATTTGGCGAGGTGATTAGCT |
| | | ATCAGCAGCTGGCAGCACTGGCAGGTAATCCGGCAGCA |
| | | ACCGCAGCAGTTAAAACCGCACTGAGCGGCAATCCGGT |
| | | TCCGATTCTGATTCCGTGTCATCGTGTTGTTAGCAGCAG |
| | | CGGTGCAGTTGGTGGTTATGAAGGTGGTCTGGCAGTTA |
| | | AAGAATGGCTGCTGGCACATGAAGGCCATCGTCTGGGC |
| | | AAACCTGGTTTAGGCGGTAGCAGTGGTGCAAGCCCTGC |
| | | AGCACCGGCTCCGGCAAGTCCAGCAGCACCAGCACCTA |
| | | GCGCACCAGCCGGTGGTAACAAATTTAACAAAGAAATG |
| | | CGTAACGCCTATTGGGAAATTGCACTGCTGCCGAATCT |
| | | GAATAATCAGCAGAAACGTGCATTTATCCGTAGCCTGT |
| | | ATGATGATCCGAGCCAGAGCGCAAATCTGCTGGCCGAA |
| | | GCAAAGAAACTGAATGATGCACAGGCACCGAAATAAG |
| | | TCGACCGGCTGCTAACAAAGCCCGAAAGGAAGCTGAGT |
| | | TGGCTGCTGCCACCGCTGAGCAATAACTAGCATAACCC |
| | | CTTGGGGCCTCTAAACGGGTCTTGAGGGGTTTTTTG |
| Transmembrane | 4 | TAATACGACTCACTATAGGGAGACCACAACGGTTTCCC |
| αHER2 | | TCTAGAAATAATTTTGTTTAACTTTAAGAAGGAGATATA |
| affibody | | CATATGGACTATAAAGACGATGACGATAAACACATGGG |
| [10] | | TAGCACCCGTACCGAAATTATCACCCGTCTGAGCTTTAG |
| | | CCTGCTGCTGCAGCTGGTTCTGGCAATTTTTCTGCTGGC |
| | | ACTGCTGATTGTTCTGCTGTGGCTGCTGCAACAGCTGAA |
| | | AGAACTGCTGCGTGAACTGGAACGTCTGCAGCGTGAAG |
| | | GTAGCAGTGATGAAGATGTTCGTGAGCTGCTGCGCGAA |
| | | ATTAAAGAACTGGTTGAAAACATTGTGTATCTGGTGAT |
| | | CATTATCATGGTTCTGGTGCTGGTTATTATTGCACTGGC |
| | | AGTTCTGCAGATGTACCTGGTTCGGGAACTGAAACGTC |
| | | AGGATGGTAGCTCAGGATCCGCAAGTCCGGCAGCACCG |
| | | GCACCGGCATCACCAGCAGCTCCAGCACCTAGTGCACC |
| | | GGCAGGCGGTAACAAATTTAACAAAGAAATGCGTAACG |
| | | CCTATTGGGAAATTGCACTGCTGCCGAATCTGAATAATC |
| | | AGCAGAAACGTGCATTTATCCGTAGCCTGTATGATGAT |
| | | CCGAGCCAGAGCGCAAATCTGCTGGCCGAAGCAAAGA |
| | | AACTGAATGATGCACAGGCACCGAAATAAGTCGACCGG |
| | | CTGCTAACAAAGCCCGAAAGGAAGCTGAGTTGGCTGCT |
| | | GCCACCGCTGAGCAATAACTAGCATAACCCCTTGGGGC |
| | | CTCTAAACGGGTCTTGAGGGGTTTTTTG |
| SNAP-αEGFR | 5 | TAATACGACTCACTATAGGGAGACCACAACGGTTTCCC |
| miniprotein | | TCTAGAAATAATTTTGTTTAACTTTAAGAAGGAGATATA |
| [11] | | CATATGGACTATAAAGACGATGACGATAAACACATGGG |
| | | TAGCATGGACAAAGATTGCGAAATGAAACGTACCACCC |
| | | TGGATAGCCCGCTGGGCAAACTGGAACTGAGCGGCTGC |
| | | GAACAGGGCCTGCATGAAATTAAACTGCTGGGTAAAGG |
| | | CACCAGCGCGGCCGATGCGGTTGAAGTTCCGGCCCCGG |
| | | CCGCCGTGCTGGGTGGTCCGGAACCGCTGATGCAGGCG |
| | | ACCGCGTGGCTGAACGCGTATTTTCATCAGCCGGAAGC |
| | | GATTGAAGAATTTCCGGTTCCGGCGCTGCATCATCCGGT |
| | | GTTTCAGCAGGAGAGCTTTACCCGTCAGGTGCTGTGGA |
| | | AACTGCTGAAAGTGGTTAAATTTGGCGAAGTGATTAGC |
| | | TATCAGCAGCTGGCGGCCCTGGCGGGTAATCCGGCGGC |
| | | CACCGCCGCCGTTAAAACCGCGCTGAGCGGTAACCCGG |
| | | TGCCGATTCTGATTCCGTGCCATCGTGTGGTTAGCTCTA |
| | | GCGGTGCGGTTGGCGGTTATGAAGGTGGTCTGGCGGTG |
| | | AAAGAGTGGCTGCTGGCCCATGAAGGTCATCGTCTGGG |
| | | TAAACCGGGTCTGGGAGGTAGCTCAGGAGCAAGTCCGG |
| | | CAGCACCGGCACCGGCATCACCAGCAGCTCCAGCACCT |
| | | AGTGCACCGGCAGGCGGTGATCATTGGGAAGAAGTTTT |
| | | TCGTTGGGCATTAGAACATCTGCAAGAGGCAACCCAGC |
| | | AGAATGATCCGCAGAAAGCAAAGAAAATTCTGGAAGA |
| | | GGCACATAAATGGCTGCGTCGTGAACTGAGCGAAGAAG |
| | | AAGCACGCGCAGTTGTTCGTTGGCTGAAACAGCTGGTT |
| | | GATCGCGAACTGAGCTAAGTCGACCGGCTGCTAACAAA |
| | | GCCCGAAAGGAAGCTGAGTTGGCTGCTGCCACCGCTGA |
| | | GCAATAACTAGCATAACCCCTTGGGGCCTCTAAACGGG |
| | | TCTTGAGGGGTTTTTTG |
| SNAP-αPDL1 | 6 | TAATACGACTCACTATAGGGAGACCACAACGGTTTCCC |
| miniprotein | | TCTAGAAATAATTTTGTTTAACTTTAAGAAGGAGATATA |
| [12] | | CATATGGACTATAAAGACGATGACGATAAACACATGGG |
| | | TAGCATGGACAAAGATTGCGAAATGAAACGTACCACCC |
| | | TGGATAGCCCGCTGGGCAAACTGGAACTGAGCGGCTGC |
| | | GAACAGGGCCTGCATGAAATTAAACTGCTGGGTAAAGG |
| | | CACCAGCGCGGCCGATGCGGTTGAAGTTCCGGCCCCGG |
| | | CCGCCGTGCTGGGTGGTCCGGAACCGCTGATGCAGGCG |
| | | ACCGCGTGGCTGAACGCGTATTTTCATCAGCCGGAAGC |
| | | GATTGAAGAATTTCCGGTTCCGGCGCTGCATCATCCGGT |
| | | GTTTCAGCAGGAGAGCTTTACCCGTCAGGTGCTGTGGA |
| | | AACTGCTGAAAGTGGTTAAATTTGGCGAAGTGATTAGC |
| | | TATCAGCAGCTGGCGGCCCTGGCGGGTAATCCGGCGGC |
| | | CACCGCCGCCGTTAAAACCGCGCTGAGCGGTAACCCGG |
| | | TGCCGATTCTGATTCCGTGCCATCGTGTGGTTAGCTCTA |
| | | GCGGTGCGGTTGGCGGTTATGAAGGTGGTCTGGCGGTG |
| | | AAAGAGTGGCTGCTGGCCCATGAAGGTCATCGTCTGGG |
| | | TAAACCGGGTCTGGGAGGTAGCTCAGGAGCAAGTCCGG |
| | | CAGCACCGGCACCGGCATCACCAGCAGCTCCAGCACCT |
| | | AGTGCACCGGCAGGCGGTATTCAGATCCGCGAATATAA |
| | | ACGTTGTGGTCAGGATGAAGAACGTGTTCGTCGTGAAT |
| | | GTAAAGAACGTGGTGAACGTCAGAATTGCCATTATGTG |
| | | ATTCATAAAGAAGGCAACTGTTACGTTTGCGGCATTATT |
| | | TGTCTGTAAGTCGACCGGCTGCTAACAAAGCCCGAAAG |
| | | GAAGCTGAGTTGGCTGCTGCCACCGCTGAGCAATAACT |
| | | AGCATAACCCCTTGGGGCCTCTAAACGGGTCTTGAGGG |
| | | GTTTTTTG |
| SNAP-αCD3 | 7 | TAATACGACTCACTATAGGGAGACCACAACGGTTTCCC |
| miniprotein | | TCTAGAAATAATTTTGTTTAACTTTAAGAAGGAGATATA |
| [11] | | CATATGGACTATAAAGACGATGACGATAAACACATGGG |
| | | TAGCATGGACAAAGATTGCGAAATGAAACGTACCACCC |
| | | TGGATAGCCCGCTGGGCAAACTGGAACTGAGCGGCTGC |
| | | GAACAGGGCCTGCATGAAATTAAACTGCTGGGTAAAGG |
| | | CACCAGCGCGGCCGATGCGGTTGAAGTTCCGGCCCCGG |
| | | CCGCCGTGCTGGGTGGTCCGGAACCGCTGATGCAGGCG |
| | | ACCGCGTGGCTGAACGCGTATTTTCATCAGCCGGAAGC |
| | | GATTGAAGAATTTCCGGTTCCGGCGCTGCATCATCCGGT |
| | | GTTTCAGCAGGAGAGCTTTACCCGTCAGGTGCTGTGGA |
| | | AACTGCTGAAAGTGGTTAAATTTGGCGAAGTGATTAGC |
| | | TATCAGCAGCTGGCGGCCCTGGCGGGTAATCCGGCGGC |
| | | CACCGCCGCCGTTAAAACCGCGCTGAGCGGTAACCCGG |
| | | TGCCGATTCTGATTCCGTGCCATCGTGTGGTTAGCTCTA |
| | | GCGGTGCGGTTGGCGGTTATGAAGGTGGTCTGGCGGTG |
| | | AAAGAGTGGCTGCTGGCCCATGAAGGTCATCGTCTGGG |
| | | TAAACCGGGTCTGGGAGGTAGCTCAGGAGCAAGTCCGG |
| | | CAGCACCGGCACCGGCATCACCAGCAGCTCCAGCACCT |
| | | AGTGCACCGGCAGGCGGTAATCATATCGCCTGCGAAAT |
| | | TCATAATCCGGAAGCAGCAAAAGAAATTGCCAAAGTTG |
| | | CAAATGTTCGTCGCGTGTACTTTATCAAACAGCCTGGTA |
| | | ATCGTTATTTCGTGCTGCTGAAAAATGCAGATCCGGAA |
| | | GGTGTTAAAAAGGTGCGTAGCAAATACAATGTGCGTTG |
| | | CGTTATTCGTGAATAAGTCGACCGGCTGCTAACAAAGC |
| | | CCGAAAGGAAGCTGAGTTGGCTGCTGCCACCGCTGAGC |
| | | AATAACTAGCATAACCCCTTGGGGCCTCTAAACGGGTC |
| | | TTGAGGGGTTTTTTG |
| SNAP-αCD3 | 8 | TAATACGACTCACTATAGGGAGACCACAACGGTTTCCC |
| scFv | | TCTAGAAATAATTTTGTTTAACTTTAAGAAGGAGATATA |
| [13] | | CATATGGACTATAAAGACGATGACGATAAACATATGGG |
| | | CAGCATGGATAAAGATTGCGAAATGAAACGTACCACAC |
| | | TGGATAGTCCGCTGGGTAAACTGGAACTGAGCGGTTGT |
| | | GAACAGGGTCTGCATGAAATTAAACTGTTAGGTAAAGG |
| | | CACCAGCGCAGCAGATGCAGTTGAAGTTCCGGCACCGG |
| | | CAGCAGTTTTAGGTGGTCCGGAACCGCTGATGCAGGCA |
| | | ACCGCATGGCTGAATGCATATTTTCATCAGCCGGAAGC |
| | | CATTGAAGAATTTCCGGTTCCTGCACTGCATCATCCGGT |
| | | TTTTCAGCAAGAAAGCTTTACCCGTCAGGTTCTGTGGAA |
| | | ACTGCTGAAAGTTGTTAAATTTGGCGAGGTGATTAGCT |
| | | ATCAGCAGCTGGCAGCACTGGCAGGTAATCCGGCAGCA |
| | | ACCGCAGCAGTTAAAACCGCACTGAGCGGCAATCCGGT |
| | | TCCGATTCTGATTCCGTGTCATCGTGTTGTTAGCAGCAG |
| | | CGGTGCAGTTGGTGGTTATGAAGGTGGTCTGGCAGTTA |
| | | AAGAATGGCTGCTGGCACATGAAGGCCATCGTCTGGGC |
| | | AAACCTGGTTTAGGCGGTAGCAGTGGTGCAAGCCCTGC |
| | | AGCACCGGCTCCGGCAAGTCCAGCAGCACCAGCACCTA |
| | | GCGCACCAGCCGGTGGTGATATCAAACTGCAGCAGAGC |
| | | GGTGCAGAACTGGCACGTCCGGGTGCAAGCGTTAAAAT |
| | | GAGCTGTAAAACCAGCGGTTATACCTTTACACGTTATAC |
| | | CATGCATTGGGTTAAACAGCGTCCTGGTCAAGGTCTGG |
| | | AATGGATTGGTTATATCAATCCGAGCCGTGGTTATACCA |
| | | ATTACAACCAGAAATTCAAAGACAAAGCAACCCTGACC |
| | | ACCGATAAAAGCAGCAGCACCGCCTATATGCAGCTGAG |
| | | CAGCCTGACCAGCGAAGATAGCGCAGTTTATTACTGTG |
| | | CACGCTATTATGATGATCACTATTGCCTGGATTATTGGG |
| | | GTCAGGGCACCACACTGACCGTTAGCAGCGTTGAAGGT |
| | | GGTAGCGGTGGTTCAGGTGGTAGTGGCGGTAGTGGTGG |
| | | TGTTGATGATATTCAGCTGACCCAGAGTCCGGCAATTAT |
| | | GAGCGCAAGTCCGGGTGAAAAAGTTACCATGACCTGTC |
| | | GTGCCAGCAGCAGCGTTAGCTATATGAATTGGTATCAG |
| | | CAGAAAAGCGGTACAAGCCCGAAACGTTGGATTTATGA |
| | | TACCAGCAAAGTTGCAAGCGGTGTTCCGTATCGTTTTAG |
| | | CGGTAGCGGTTCAGGCACCAGCTATAGTCTGACCATTA |
| | | GCTCAATGGAAGCAGAAGATGCAGCAACCTATTATTGT |
| | | CAGCAGTGGTCAAGCAATCCGCTGACCTTTGGTGCAGG |
| | | TACAAAACTGGAACTGAAATAAGTCGACCGGCTGCTAA |
| | | CAAAGCCCGAAAGGAAGCTGAGTTGGCTGCTGCCACCG |
| | | CTGAGCAATAACTAGCATAACCCCTTGGGGCCTCTAAA |
| | | CGGGTCTTGAGGGGTTTTTTG |
| SNAP-αCD2 | 9 | CAATCCGCCCTCACTACAACCGTAATACGACTCACTATA |
| scFv | | GGGAGACCACAACGGTTTCCCTCTAGAAATAATTTTGTT |
| [14] | | TAACTTTAAGAAGGAGATATACATATGGACTATAAAGA |
| | | CGATGACGATAAACATATGGGCAGCATGGATAAAGATT |
| | | GCGAAATGAAACGTACCACACTGGATAGTCCGCTGGGT |
| | | AAACTGGAACTGAGCGGTTGTGAACAGGGTCTGCATGA |
| | | AATTAAACTGTTAGGTAAAGGCACCAGCGCAGCAGATG |
| | | CAGTTGAAGTTCCGGCACCGGCAGCAGTTTTAGGTGGT |
| | | CCGGAACCGCTGATGCAGGCAACCGCATGGCTGAATGC |
| | | ATATTTTCATCAGCCGGAAGCCATTGAAGAATTTCCGGT |
| | | TCCTGCACTGCATCATCCGGTTTTTCAGCAAGAAAGCTT |
| | | TACCCGTCAGGTTCTGTGGAAACTGCTGAAAGTTGTTAA |
| | | ATTTGGCGAGGTGATTAGCTATCAGCAGCTGGCAGCAC |
| | | TGGCAGGTAATCCGGCAGCAACCGCAGCAGTTAAAACC |
| | | GCACTGAGCGGCAATCCGGTTCCGATTCTGATTCCGTGT |
| | | CATCGTGTTGTTAGCAGCAGCGGTGCAGTTGGTGGTTAT |
| | | GAAGGTGGTCTGGCAGTTAAAGAATGGCTGCTGGCACA |
| | | TGAAGGCCATCGTCTGGGCAAACCTGGTTTAGGCGGTA |
| | | GCAGTGGTGCAAGCCCTGCAGCACCGGCTCCGGCAAGT |
| | | CCAGCAGCACCAGCACCTAGCGCACCAGCCGGTGGTAA |
| | | CATTATGATGACCCAGAGTCCGAGCAGCCTGGCAGTTA |
| | | GTGCCGGTGAAAAAGTTACCATGACCTGTAAAAGCAGC |
| | | CAGAGCGTTCTGTATAGCAGCAATCAGAAAAACTATCT |
| | | GGCATGGTATCAGCAGAAACCGGGTCAGAGCCCGAAAC |
| | | TGCTGATTTATTGGGCAAGCACCCGTGAAAGCGGTGTT |
| | | CCGGATCGTTTTACCGGTAGCGGTAGCGGCACCGATTTT |
| | | ACCCTGACCATTAGCAGCGTTCAGCCGGAAGATCTGGC |
| | | AGTGTATTATTGTCATCAGTATCTGAGCAGCCATACCTT |
| | | TGGTGGTGGCACCAAACTGGAAATTAAACGCGGTGGTG |
| | | GTGGTAGTGGTGGCGGTGGTTCAGGCGGTGGCGGTAGC |
| | | CAGCTGCAGCAGCCAGGTGCAGAACTGGTTCGTCCGGG |
| | | TAGCAGCGTGAAACTGAGCTGTAAAGCAAGCGGTTATA |
| | | CCTTTACACGCTATTGGATTCATTGGGTTAAACAGCGTC |
| | | CGATTCAAGGTCTGGAATGGATTGGTAATATTGATCCG |
| | | AGCGATAGCGAAACCCATTACAACCAGAAATTCAAAGA |
| | | TAAAGCAACCCTGACCGTGGATAAAAGCAGTGGCACCG |
| | | CCTATATGCAGCTGAGCAGTCTGACCAGCGAAGATAGC |
| | | GCAGTTTATTACTGTGCAACCGAAGATCTGTATTACGCC |
| | | ATGGAATATTGGGGTCAGGGCACCAGCGTTACCGTTAG |
| | | CAGCTAAGTCGACCGGCTGCTAACAAAGCCCGAAAGGA |
| | | AGCTGAGTTGGCTGCTGCCACCGCTGAGCAATAACTAG |
| | | CATAACCCCTTGGGGCCTCTAAACGGGTCTTGAGGGGT |
| | | TTTTTGCTACTCTGGCGTCGATGAGGGA |

### Cell Culture

All cell culture media was purchased from ThermoFisher. Jurkat cells were purchased from ATCC and cultured with RPMI supplemented with GlutaMAX, 10% fetal bovine serum (FBS), and 1% penicillin streptomycin (penstrep). MDA-MB-231 and SKBR3 cells were gifted from the Mrksich lab (ATCC) and cultured with DMEM supplemented with GlutaMAX, 10% FBS, and 1% penstrep. NFAT-GFP Jurkat reporter cells were created using lentivirus (BPS Bioscience) were created using the manufacturer's instructions. Cells were cultured at 37 °C 5% CO₂.

### Cell-Free Protein Expression and Conjugation

Protein expression was performed with the PURExpress In Vitro Protein Synthesis kit (E6800, NEB) according to the manufacturer's instructions. Reactions were assembled on ice with 3.3 nM plasmid and PURExpress Disulfide Bond Enhancer and allowed to progress at 30 °C for 3 hours (or up to overnight). To conjugate transmembrane-fused HER2 affibody to liposomes, 10 mM liposomes were included in the cell-free reaction mixture as transmembrane proteins cotranslationally insert into vesicle membranes.

Following expression, chimeric SNAP proteins were conjugated to membrane-based materials by incubating cell-free reaction mixtures with vesicles at a specified ratio for 2 hours at room temperature or overnight at 4 °C. A standard ratio used was 4 μL of cell-free reaction (protein yields found in FIG. 8) to 2 μL of 10 mM lipid.

### Protein Quantification by Gel Electrophoresis

Chimeric SNAP protein yields were determined by SDS-PAGE analysis. Cell-free expressed proteins were labeled with SNAP547 per the manufacturer's instructions. Briefly, 1 μL of cell-free protein synthesis reaction was incubated with 5 M SNAP547 for 30 minutes at 37 °C. Cell-free samples and purified, labeled SNAP were then run on a 12% Mini-PROTEAN TGX Precast Gel (Biorad). Gels were imaged using an Azure Sapphire Imager and proteins were quantified by comparing cell-free protein fluorescence to purified protein standards using ImageJ.

### Preparation of Membrane-Based Materials

Liposomes and polymersomes were prepared using the thin hydration method. Briefly, lipid or polymer was deposited into a glass vial and dried with nitrogen and placed under vacuum for 2 hours. Films were then rehydrated in PBS heated at 60 °C for a minimum of 2 hours, and up to overnight. Vesicles were then briefly vortexed and extruded 21× through a 100 nm polycarbonate filter.

Lipid nanoparticles were prepared by dissolving lipids in ethanol and mixing with 7.5 mM citrate buffer. The mixture was then sonicated for 15 minutes in a bath sonicator and dialysis was performed in PBS for 2 hours, and up to overnight, at 4 °C using 3.5 k MWCO Slide-A-Lyzer MINI dialysis devices. For lipid nanoparticles encapsulating mRNA, mRNA encapsulation efficiency was measured using a RiboGreen Assay (Invitrogen) according to the manufacturer's instructions in the presence and absence of 0.1% Triton-X.

### Assessment of In Vitro Binding

Adherent cells were removed from culture plates using TrypLE (ThermoFisher) to minimize disruption of cell surface markers. Cells were washed with flow buffer (PBS with 1% FBS and 2.5 μM EDTA, sterile filtered) and resuspended at 100,000 cells in 100 μL flow buffer. For surface confirmation using commercial antibodies, 1 μL of 200 μg/mL isotype IgG was added 15 minutes prior to antibody incubation at room temperature, and then 5 μL of 0.5 mg/ml commercial antibodies were for 30 minutes at room temperature. For fluorescently labeled cell free expressed proteins and nanoparticles, washed cells were incubated with 4 μL of protein or protein on vesicles unless indicated for 30 minutes. Cells were then washed 2 times with flow buffer, resuspended in flow buffer, and analyzed using flow cytometry (BD LSRFortessa II). Rhodamine, PE, and SNAP547 were read with 550 nm excitation, 582/15 emission, and Cy5.5 was read with 690 nm excitation, 750/50 (710 long pass) emission.

### T-Cell Binding and Activation Experiments

NFAT-Jurkat cells in full media were added to a 96 well plate at 100,000 cells per well in 100 μL. Proteins and vesicles were then added to the cells, and the cells were incubated in the incubator at 37 °C for 6 hours to allow for activation. After incubation, cells were washed 2× in flow buffer and resuspended in flow buffer. GFP expression was measured using flow cytometry. GFP was read with 488 nm excitation, 530/30 (505 long pass) emission

mRNA Delivery to T-Cells

Lipid nanoparticles were produced as described above, by rehydrating lipids with mRNA encoding for firefly luciferase or eGFP. Following preparation, mRNA encapsulation efficiency was measured via a RiboGreen Assay and cell-free proteins were conjugated to LNPs. LNPs were then added to 1E6 Jurkat cells and incubated for 24 hours. For experiments in which firefly luciferase was delivered, cell viability and luciferase expression were evaluated using the ONE-Glo+Tox Luciferase Reporter and Cell Viability Assay (Promega) according to the manufacturer's instructions. For eGFP mRNA experiments, expression was evaluated using microscopy and flow cytometry.

### REFERENCES

[1] R. Tenchov, R. Bird, A. E. Curtze, Q. Zhou, ACS Nano 2021, 15, 16982.
[2] M. J. Mitchell, M. M. Billingsley, R. M. Haley, M. E. Wechsler, N. A. Peppas, R. Langer, Nat Rev Drug Discov 2021, 20, 101.
[3] Z. Zhao, A. Ukidve, J. Kim, S. Mitragotri, Cell 2020, 181, 151.
[4] K. R. Rhodes, J. J. Green, Mol Immunol 2018, 98, 13.
[5] H. Moulahoum, F. Ghorbanizamani, F. Zihnioglu, S. Timur, Bioconjug Chem 2021, 32, 1491.
[6] I. Tomé, V. Francisco, H. Fernandes, L. Ferreira, APL Bioeng 2021, 5, 031511.
[7] I. Menon, M. Zaroudi, Y. Zhang, E. Aisenbrey, L. Hui, Mater Today Adv 2022, 16, 100299.
[8] C. Tuckey, H. Asahara, Y. Zhou, S. Chong, Current protocols in molecular biology/edited by Frederick M. Ausubel . . . [et al.] 2014, 108, 16.31.1.
[9] A. K. Rudd, J. M. Valls Cuevas, N. K. Devaraj, J Am Chem Soc 2015, 137, 4884.
[10] S. B. Lee, M. Hassan, R. Fisher, O. Chertov, V. Chernomordik, G. Kramer-Marek, A. Gandjbakhche, J. Capala, Clinical Cancer Research 2008, 14, 3840.
[11] L. Cao, B. Coventry, I. Goreshnik, B. Huang, W. Sheffler, J. S. Park, K. M. Jude, I. Marković, R. U. Kadam, K. H. G. Verschueren, K. Verstraete, S. T. R. Walsh, N. Bennett, A. Phal, A. Yang, L. Kozodoy, M. DeWitt, L. Picton, L. Miller, E. M. Strauch, N. D. DeBouver, A. Pires, A. K. Bera, S. Halabiya, B. Hammerson, W. Yang, S. Bernard, L. Stewart, I. A. Wilson, H. Ruohola-Baker, J. Schlessinger, S. Lee, S. N. Savvides, K. C. Garcia, D. Baker, Nature 2022 605:7910 2022, 605, 551.
[12] H. Yin, X. Zhou, Y. H. Huang, G. J. King, B. M. Collins, Y. Gao, D. J. Craik, C. K. Wang, J Am Chem Soc 2021, 143, 18536.
[13] “BLINATUMOMAB,” can be found under https://drugs.ncats.io/substance/4FR53SIF3A #details, n.d.
[14] R. J. Connelly, M. S. Hayden, J. K. Scholler, T. T. Tsu, B. Dupont, J. A. Ledbetter, S. B. Kanner, Int Immunol 1998, 10, 1863.
[15] L. Ekerljung, H. Wällberg, A. Sohrabian, K. Andersson, M. Friedman, F. Y. Frejd, S. Stähl, L. Gedda, Bioconjug Chem 2012, 23, 1802.
[16] J. L. Dopp, N. F. Reuel, Biochem Eng J 2020, 164, 107790.
[17] M. J. Mitchell, M. M. Billingsley, R. M. Haley, M. E. Wechsler, N. A. Peppas, R. Langer, Nat Rev Drug Discov 2021, 20, 101.
[18] P. M. Cevaal, A. Ali, E. Czuba-Wojnilowicz, J. Symons, S. R. Lewin, C. Cortez-Jugo, F. Caruso, ACS Nano 2021, 15, 3736.
[19] P. Lu, D. Min, F. DiMaio, K. Y. Wei, M. D. Vahey, S. E. Boyken, Z. Chen, J. A. Fallas, G. Ueda, W. Sheffler, V. K. Mulligan, W. Xu, J. U. Bowie, D. Baker, Science (1979) 2018, 359, 1042.
[20] J. A. Peruzzi, J. Steinkühler, T. Q. Vu, T. F. Gunnels, P. Lu, D. Baker, N. P. Kamat, bioRxiv 2022, 2022.06.01.494374.
[21] H. Y. Lee, E. Contreras, A. C. Register, Q. Wu, K. Abadie, K. Garcia, P. Y. Wong, G. Jiang, Sci Rep 2019, 9, 3900.
[22] J. J. Lin, G. P. O'Donoghue, K. B. Wilhelm, M. P. Coyle, S. T. Low-Nam, N. C. Fay, K. N. Alfieri, J. T. Groves, Biophys J 2020, 118, 2879.
[23] T. Q. Vu, J. A. Peruzzi, L. E. Sant'Anna, E. W. Roth, N. P. Kamat, Nano Lett 2022, acs. nanolett. 1c04365.
[24] P. Demetriou, E. Abu-Shah, S. Valvo, S. McCuaig, V. Mayya, A. Kvalvaag, T. Starkey, K. Korobchevskaya, L. Y. W. Lee, M. Friedrich, E. Mann, M. A. Kutuzov, M. Morotti, N. Wietek, H. Rada, S. Yusuf, J. Afrose, A. Siokis, P. Allan, T. Ambrose, C. Arancibia, A. Bailey, E. Barnes, E. Bird-Lieberman, J. Bornschein, O. Brain, B. Braden, J. Collier, J. Cobbold, E. Culver, J. East, L. Howarth, P. Klenerman, S. Leedham, R. Palmer, M. Pavlides, F. Powrie, A. Rodrigues, J. Satsangi, A. Simmons, P. Sullivan, H. Uhlig, A. Walsh, M. Meyer-Hermann, A. A. Ahmed, D. Depoil, M. L. Dustin, Nat Immunol 2020, 21, 1232.
[25] J. Leitner, D. Herndler-Brandstetter, G. J. Zlabinger, B. Grubeck-Loebenstein, P. Steinberger, The Journal of Immunology 2015, 195, 477.
[26] L. Zhang, S. Song, X. Jin, X. Wan, K. A. Shahzad, W. Pei, C. Zhao, C. Shen, Cancer Immunol Res 2019, 7, 1188.
[27] K. Andrews, A. A. J. Hamers, X. Sun, G. Neale, K. Verbist, P. Tedrick, K. E. Nichols, S. Pereira, D. E. Geraghty, A. B. Pillai, Cytotherapy 2020, 22, 276.
[28] A. Kheirolomoom, A. J. Kare, E. S. Ingham, R. Paulmurugan, E. R. Robinson, M. Baikoghli, M. Inayathullah, J. W. Seo, J. Wang, B. Z. Fite, B. Wu, S. K. Tumbale, M. N. Raie, R. H. Cheng, L. Nichols, A. D. Borowsky, K. W. Ferrara, Biomaterials 2022, 281, 121339.
[29] D. J. A. Crommelin, P. van Hoogevest, G. Storm, Journal of Controlled Release 2020, 318, 256.
[30] J. G. Rurik, I. Tombacz, A. Yadegari, P. O. Méndez Fernández, S. v. Shewale, L. Li, T. Kimura, O. Y. Soliman, T. E. Papp, Y. K. Tam, B. L. Mui, S. M. Albelda, E. Puré, C. H. June, H. Aghajanian, D. Weissman, H. Parhiz, J. A. Epstein, Science (1979) 2022, 375, 91.
[31] B. C. Bundy, M. J. Franciszkowicz, J. R. Swartz, Biotechnol Bioeng 2008, 100, 28.
[32] K. G. Patel, J. R. Swartz, Bioconjug Chem 2011, 22, 376.
[33] J. C. Stark, T. Jaroentomeechai, T. D. Moeller, J. M. Hershewe, K. F. Warfel, B. S. Moricz, A. M. Martini, R. S. Dubner, K. J. Hsu, T. C. Stevenson, B. D. Jones, M. P. DeLisa, M. C. Jewett, Sci Adv 2021, 7, DOI 10.1126/SCIADV.ABE9444/SUPPL_FILE/ABE9444_SM.PDF.
[34] K. F. Warfel, A. Williams, D. A. Wong, S. E. Sobol, P. Desai, J. Li, Y.-F. Chang, M. P. Delisa, A. S. Karim, M. C. Jewett, R. Frederick, bioRxiv 2022, 2022.08.10.503507.
[35] Y. Lu, J. P. Welsh, J. R. Swartz, Proc Natl Acad Sci USA 2014, 111, 125.
[36] G. Kanter, J. Yang, A. Voloshin, S. Levy, J. R. Swartz, R. Levy, Blood 2007, 109, 3393.
[37] A. C. Hunt, B. Vögeli, W. K. Kightlinger, D. J. Yoesep, A. Krüger, M. C. Jewett, bioRxiv 2021, 2021.11.04.467378.
[38] A. Pandi, D. Adam, A. Zare, V. T. Trinh, S. L. Schaefer, M. Wiegand, B. Klabunde, E. Bobkova, M. Kushwaha, Y. Foroughijabbari, P. Braun, C. Spahn, C. Preußer, E. P. von Strandmann, H. B. Bode, H. von Buttlar, W. Bertrams, A. L. Jung, F. Abendroth, B. Schmeck, G. Hummer, O. Vázquez, T. J. Erb, bioRxiv 2022, 14, 2022.11.19.517184.
[39] A. O. Robinson, O. M. Venero, K. P. Adamala, Curr Opin Chem Biol 2021, 64, 165.
[40] K. P. Adamala, D. A. Martin-Alarcon, K. R. Guthrie-Honea, E. S. Boyden, Nature Chemistry 2016 9:5 2016, 9, 431.
[41] J. A. Peruzzi, N. R. Galvez, N. P. Kamat, bioRxiv 2022, 2022.10.30.514420.
[42] Ö. D. Toparlak, J. Zasso, S. Bridi, M. D. Serra, P. Macchi, L. Conti, M. L. Baudet, S. S. Mansy, Sci Adv 2020, 6, 4920.
[43] O. Adir, M. R. Albalak, R. Abel, L. E. Weiss, G. Chen, A. Gruber, O. Staufer, Y. Kurman, I. Kaminer, J. Shklover, J. Shainsky-Roitman, I. Platzman, L. Gepstein, Y. Shechtman, B. A. Horwitz, A. Schroeder, Nat Commun 2022, 13, DOI 10.1038/S41467-022-29871-8.
[44] M. M. Hasani-Sadrabadi, F. S. Majedi, M. L. Miller, T. J. Thauland, L. S. Bouchard, S. Li, M. J. Butte, Mater Horiz 2020, 7, 3028.
[45] G. Chen, R. Levin, S. Landau, M. Kaduri, O. Adir, I. Ianovici, N. Krinsky, O. Doppelt-Flikshtain, J. Shklover, J. Shainsky-Roitman, S. Levenberg, A. Schroeder, Proc Natl Acad Sci USA 2022, 119, DOI 10.1073/pnas.2207525119.
[46] Ö. D. Toparlak, J. Zasso, S. Bridi, M. D. Serra, P. Macchi, L. Conti, M. L. Baudet, S. S. Mansy, Sci Adv 2020, 6, 4920.
[47] N. Krinsky, M. Kaduri, A. Zinger, J. Shainsky-Roitman, M. Goldfeder, I. Benhar, D. Hershkovitz, A. Schroeder, Adv Healthc Mater 2018, 7, 1870038.

1. A method for functionalizing a nanocarrier with a functional protein, the method comprising:
assembling the nanocarrier with a modified lipid;
incubating the assembled nanocarrier with a cell-free protein synthesis system and
a nucleic acid template encoding a fusion protein, the fusion protein comprising:
a protein tag that conjugates to the modified lipid; and
the functional protein.

2. The method of claim 1, wherein the protein tag is a self-labeling enzyme.

3. The method of claim 2, wherein the modified lipid comprises benzylguanine and the self-labeling enzyme is a SNAP-tag.

4. The method of any one of claims 1-3, wherein the functional protein is selected from an antibody fragment, a de novo designed protein, and a receptor.

5. The method of any one of claims 1-4, wherein the nanocarrier is a liposome, a polymersome, a lipid nanoparticle, an extracellular vesicle, or an outer membrane vesicle.

6. A method for functionalizing a nanocarrier with a functional protein, the method comprising:
incubating the nanocarrier with a cell-free protein synthesis system and a nucleic acid template encoding a fusion protein, the fusion protein comprising:
a transmembrane domain; and
the functional protein.

7. The method of claim 6, wherein the functional protein is selected from an antibody fragment, a de novo designed protein, and a receptor.

8. A method for delivering a cargo to a cell, the method comprising:
encapsulating the cargo into a nanocarrier;
functionalizing the nanocarrier according to the method of any one of claims 1-7; and
dosing the cell with the functionalized nanocarrier;
wherein the cell comprises a surface protein that binds to the functional protein.

9. The method of claim 8, wherein the cargo comprises a polynucleotide, a polypeptide, an active pharmaceutical ingredient, or a therapeutic agent.

10. The method of claim 9, wherein the cargo is an mRNA molecule.

11. A method for generating an artificial antigen presenting cell, the method comprising:
functionalizing a nanocarrier according to the method of claim 1 or claim 6,
wherein the functional protein is an antigen.

12. The method of claim 11, wherein the antigen is an scFv.

13. The method of claim 12, wherein the scFv is specific for T lymphocytes.

14. The method of any one of claims 11-13, wherein the nanocarrier comprises one or more additional nucleic acid templates, wherein each nucleic acid template of the nanocarrier encodes a different functional protein.

15. A method for activating a T lymphocyte, the method comprising:
incubating the T lymphocyte with the artificial antigen presenting cell of any one of claims 11-14.

16. A composition comprising:
a nanocarrier comprising a modified lipid;
a cell-free protein synthesis system; and
a nucleic acid template encoding a fusion protein, the fusion protein comprising:
a protein tag that conjugates to the modified lipid; and
a functional protein.

17. The composition of claim 16, wherein the protein tag is a self-labeling enzyme.

18. The composition of claim 17, wherein the modified lipid comprises benzylguanine and the self-labeling enzyme is a SNAP-tag.

19. The composition of any one of claims 16-18, wherein the functional protein is selected from an antibody fragment, a de novo designed protein, and a receptor.

20. The composition of any one of claims 16-19, wherein the nanocarrier is a liposome, a polymersome, or a lipid nanoparticle.

21. The composition of any one of claims 16-20, wherein the nanocarrier further comprises a cargo molecule.

22. The composition of claim 21, wherein the cargo molecule comprises a polynucleotide, a polypeptide, an active pharmaceutical ingredient, or a therapeutic agent.

23. A composition comprising:
a nanocarrier;
a cell-free protein synthesis system; and
a nucleic acid template encoding a fusion protein, the fusion protein comprising:
a transmembrane domain; and
a functional protein.

24. The composition of claim 23, wherein the functional protein is selected from an antibody fragment, a de novo designed protein, and a receptor.

25. The composition of claim 23 or claim 24, wherein the nanocarrier is a liposome, a polymersome, or a lipid nanoparticle.

26. The composition of any one of claims 23-25, wherein the nanocarrier further comprises a cargo molecule.

27. The composition of claim 26, wherein the cargo molecule comprises a polynucleotide, a polypeptide, an active pharmaceutical ingredient, or a therapeutic agent.


SEQUENCE LISTING (9 sequences)

SEQ ID NO: 1 - DNA, 1576 bp, synthetic construct
  source 1..1576: mol_type = other DNA
taatacgactcactatagggagaccacaacggtttccctctagaaataattttgtttaactttaagaaggagatatacatatggactataaagacgatgacgataaacacatgggtagcatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggaggtagctcaggagcaagtccggcagcaccggcaccggcatcaccagcagctccagcacctagtgcaccggcaggcggtatggttagcaaaggtgaagaactgtttaccggtgttgttccgattctggttgaactggatggtgatgttaatggccacaaattttcagttagcggtgaaggcgaaggtgatgcaacctatggtaaactgaccctgaaatttatctgtaccaccggcaaactgccggttccgtggccgacactggttaccacactgacctatggtgttcagtgttttagccgttatccggatcatatgaaacagcacgattttttcaaaagcgcaatgccggaaggttatgttcaagaacgtaccatcttcttcaaagatgacggcaactataaaacccgtgccgaagttaaatttgaaggtgataccctggtgaatcgcattgaactgaaaggcatcgattttaaagaggatggtaatatcctgggccacaaactggaatataattataatagccacaacgtgtacatcatggccgacaaacagaaaaatggcatcaaagtgaacttcaagatccgccataatattgaagatggttcagttcagctggcagatcactatcagcagaataccccgattggtgatggtccggttctgctgccggataatcattatctgagcacccagagcaaactgagcaaagatccgaatgaaaaacgtgatcacatggtgctgctggaatttgttaccgcagcaggtattaccttaggtatggatgaactgtacaaataagtcgaccggctgctaacaaagcccgaaaggaagctgagttggctgctgccaccgctgagcaataactagcataaccccttggggcctctaaacgggtcttgaggggttttttg

SEQ ID NO: 2 - DNA, 1252 bp, synthetic construct
  source 1..1252: mol_type = other DNA
taatacgactcactataggggaattgtgagcggataacaattcccctctagaaataattttgtttaactttaagaaggagatataccatgggtagcacccgtaccgaaattatcacccgtctgagctttagcctgctgctgcagctggttctggcaatttttctgctggcactgctgattgttctgctgtggctgctgcaacagctgaaagaactgctgcgtgaactggaacgtctgcagcgtgaaggtagcagtgatgaagatgttcgtgagctgctgcgcgaaattaaagaactggttgaaaacattgtgtatctggtgatcattatcatggttctggtgctggttattattgcactggcagttctgcagatgtacctggttcgggaactgaaacgtcaggatggtagcagcggtagcatggttagcaaaggtgaagaactgtttaccggtgttgttccgattctggttgaactggatggtgatgttaatggccacaaattttcagttagcggtgaaggcgaaggtgatgcaacctatggtaaactgaccctgaaatttatctgtaccaccggcaaactgccggttccgtggccgacactggttaccacactgacctatggtgttcagtgttttagccgttatccggatcatatgaaacagcacgattttttcaaaagcgcaatgccggaaggttatgttcaagaacgtaccatcttcttcaaagatgacggcaactataaaacccgtgccgaagttaaatttgaaggtgataccctggtgaatcgcattgaactgaaaggcatcgattttaaagaggatggtaatatcctgggccacaaactggaatataattataatagccacaacgtgtacatcatggccgacaaacagaaaaatggcatcaaagtgaacttcaagatccgccataatattgaagatggttcagttcagctggcagatcactatcagcagaataccccgattggtgatggtccggttctgctgccggataatcattatctgagcacccagagcaaactgagcaaagatccgaatgaaaaacgtgatcacatggtgctgctggaatttgttaccgcagcaggtattaccttaggtatggatgaactgtacaaataaggatccggctgctaacaaagcccgaaaggaagctgagttggctgctgccaccgctgagcaataactagcataaccccttggggcctctaaacgggtcttgaggggttttttg

SEQ ID NO: 3 - DNA, 1027 bp, synthetic construct
  source 1..1027: mol_type = other DNA
taatacgactcactatagggagaccacaacggtttccctctagaaataattttgtttaactttaagaaggagatatacatatggactataaagacgatgacgataaacacatgggcagcatggataaagattgcgaaatgaaacgtaccacactggatagtccgctgggtaaactggaactgagcggttgtgaacagggtctgcatgaaattaaactgttaggtaaaggcaccagcgcagcagatgcagttgaagttccggcaccggcagcagttttaggtggtccggaaccgctgatgcaggcaaccgcatggctgaatgcatattttcatcagccggaagccattgaagaatttccggttcctgcactgcatcatccggtttttcagcaagaaagctttacccgtcaggttctgtggaaactgctgaaagttgttaaatttggcgaggtgattagctatcagcagctggcagcactggcaggtaatccggcagcaaccgcagcagttaaaaccgcactgagcggcaatccggttccgattctgattccgtgtcatcgtgttgttagcagcagcggtgcagttggtggttatgaaggtggtctggcagttaaagaatggctgctggcacatgaaggccatcgtctgggcaaacctggtttaggcggtagcagtggtgcaagccctgcagcaccggctccggcaagtccagcagcaccagcacctagcgcaccagccggtggtaacaaatttaacaaagaaatgcgtaacgcctattgggaaattgcactgctgccgaatctgaataatcagcagaaacgtgcatttatccgtagcctgtatgatgatccgagccagagcgcaaatctgctggccgaagcaaagaaactgaatgatgcacaggcaccgaaataagtcgaccggctgctaacaaagcccgaaaggaagctgagttggctgctgccaccgctgagcaataactagcataaccccttggggcctctaaacgggtcttgaggggttttttg

SEQ ID NO: 4 - DNA, 793 bp, synthetic construct
  source 1..793: mol_type = other DNA
taatacgactcactatagggagaccacaacggtttccctctagaaataattttgtttaactttaagaaggagatatacatatggactataaagacgatgacgataaacacatgggtagcacccgtaccgaaattatcacccgtctgagctttagcctgctgctgcagctggttctggcaatttttctgctggcactgctgattgttctgctgtggctgctgcaacagctgaaagaactgctgcgtgaactggaacgtctgcagcgtgaaggtagcagtgatgaagatgttcgtgagctgctgcgcgaaattaaagaactggttgaaaacattgtgtatctggtgatcattatcatggttctggtgctggttattattgcactggcagttctgcagatgtacctggttcgggaactgaaacgtcaggatggtagctcaggatccgcaagtccggcagcaccggcaccggcatcaccagcagctccagcacctagtgcaccggcaggcggtaacaaatttaacaaagaaatgcgtaacgcctattgggaaattgcactgctgccgaatctgaataatcagcagaaacgtgcatttatccgtagcctgtatgatgatccgagccagagcgcaaatctgctggccgaagcaaagaaactgaatgatgcacaggcaccgaaataagtcgaccggctgctaacaaagcccgaaaggaagctgagttggctgctgccaccgctgagcaataactagcataaccccttggggcctctaaacgggtcttgaggggttttttg

SEQ ID NO: 5 - DNA, 1045 bp, synthetic construct
  source 1..1045: mol_type = other DNA
taatacgactcactatagggagaccacaacggtttccctctagaaataattttgtttaactttaagaaggagatatacatatggactataaagacgatgacgataaacacatgggtagcatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggaggtagctcaggagcaagtccggcagcaccggcaccggcatcaccagcagctccagcacctagtgcaccggcaggcggtgatcattgggaagaagtttttcgttgggcattagaacatctgcaagaggcaacccagcagaatgatccgcagaaagcaaagaaaattctggaagaggcacataaatggctgcgtcgtgaactgagcgaagaagaagcacgcgcagttgttcgttggctgaaacagctggttgatcgcgaactgagctaagtcgaccggctgctaacaaagcccgaaaggaagctgagttggctgctgccaccgctgagcaataactagcataaccccttggggcctctaaacgggtcttgaggggttttttg

SEQ ID NO: 6 - DNA, 1000 bp, synthetic construct
  source 1..1000: mol_type = other DNA
taatacgactcactatagggagaccacaacggtttccctctagaaataattttgtttaactttaagaaggagatatacatatggactataaagacgatgacgataaacacatgggtagcatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggaggtagctcaggagcaagtccggcagcaccggcaccggcatcaccagcagctccagcacctagtgcaccggcaggcggtattcagatccgcgaatataaacgttgtggtcaggatgaagaacgtgttcgtcgtgaatgtaaagaacgtggtgaacgtcagaattgccattatgtgattcataaagaaggcaactgttacgtttgcggcattatttgtctgtaagtcgaccggctgctaacaaagcccgaaaggaagctgagttggctgctgccaccgctgagcaataactagcataaccccttggggcctctaaacgggtcttgaggggttttttg

SEQ ID NO: 7 - DNA, 1045 bp, synthetic construct
  source 1..1045: mol_type = other DNA
taatacgactcactatagggagaccacaacggtttccctctagaaataattttgtttaactttaagaaggagatatacatatggactataaagacgatgacgataaacacatgggtagcatggacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggaggtagctcaggagcaagtccggcagcaccggcaccggcatcaccagcagctccagcacctagtgcaccggcaggcggtaatcatatcgcctgcgaaattcataatccggaagcagcaaaagaaattgccaaagttgcaaatgttcgtcgcgtgtactttatcaaacagcctggtaatcgttatttcgtgctgctgaaaaatgcagatccggaaggtgttaaaaaggtgcgtagcaaatacaatgtgcgttgcgttattcgtgaataagtcgaccggctgctaacaaagcccgaaaggaagctgagttggctgctgccaccgctgagcaataactagcataaccccttggggcctctaaacgggtcttgaggggttttttg

SEQ ID NO: 8 - DNA, 1588 bp, synthetic construct
  source 1..1588: mol_type = other DNA
taatacgactcactatagggagaccacaacggtttccctctagaaataattttgtttaactttaagaaggagatatacatatggactataaagacgatgacgataaacatatgggcagcatggataaagattgcgaaatgaaacgtaccacactggatagtccgctgggtaaactggaactgagcggttgtgaacagggtctgcatgaaattaaactgttaggtaaaggcaccagcgcagcagatgcagttgaagttccggcaccggcagcagttttaggtggtccggaaccgctgatgcaggcaaccgcatggctgaatgcatattttcatcagccggaagccattgaagaatttccggttcctgcactgcatcatccggtttttcagcaagaaagctttacccgtcaggttctgtggaaactgctgaaagttgttaaatttggcgaggtgattagctatcagcagctggcagcactggcaggtaatccggcagcaaccgcagcagttaaaaccgcactgagcggcaatccggttccgattctgattccgtgtcatcgtgttgttagcagcagcggtgcagttggtggttatgaaggtggtctggcagttaaagaatggctgctggcacatgaaggccatcgtctgggcaaacctggtttaggcggtagcagtggtgcaagccctgcagcaccggctccggcaagtccagcagcaccagcacctagcgcaccagccggtggtgatatcaaactgcagcagagcggtgcagaactggcacgtccgggtgcaagcgttaaaatgagctgtaaaaccagcggttatacctttacacgttataccatgcattgggttaaacagcgtcctggtcaaggtctggaatggattggttatatcaatccgagccgtggttataccaattacaaccagaaattcaaagacaaagcaaccctgaccaccgataaaagcagcagcaccgcctatatgcagctgagcagcctgaccagcgaagatagcgcagtttattactgtgcacgctattatgatgatcactattgcctggattattggggtcagggcaccacactgaccgttagcagcgttgaaggtggtagcggtggttcaggtggtagtggcggtagtggtggtgttgatgatattcagctgacccagagtccggcaattatgagcgcaagtccgggtgaaaaagttaccatgacctgtcgtgccagcagcagcgttagctatatgaattggtatcagcagaaaagcggtacaagcccgaaacgttggatttatgataccagcaaagttgcaagcggtgttccgtatcgttttagcggtagcggttcaggcaccagctatagtctgaccattagctcaatggaagcagaagatgcagcaacctattattgtcagcagtggtcaagcaatccgctgacctttggtgcaggtacaaaactggaactgaaataagtcgaccggctgctaacaaagcccgaaaggaagctgagttggctgctgccaccgctgagcaataactagcataaccccttggggcctctaaacgggtcttgaggggttttttg

SEQ ID NO: 9 - DNA, 1635 bp, synthetic construct
  source 1..1635: mol_type = other DNA
caatccgccctcactacaaccgtaatacgactcactatagggagaccacaacggtttccctctagaaataattttgtttaactttaagaaggagatatacatatggactataaagacgatgacgataaacatatgggcagcatggataaagattgcgaaatgaaacgtaccacactggatagtccgctgggtaaactggaactgagcggttgtgaacagggtctgcatgaaattaaactgttaggtaaaggcaccagcgcagcagatgcagttgaagttccggcaccggcagcagttttaggtggtccggaaccgctgatgcaggcaaccgcatggctgaatgcatattttcatcagccggaagccattgaagaatttccggttcctgcactgcatcatccggtttttcagcaagaaagctttacccgtcaggttctgtggaaactgctgaaagttgttaaatttggcgaggtgattagctatcagcagctggcagcactggcaggtaatccggcagcaaccgcagcagttaaaaccgcactgagcggcaatccggttccgattctgattccgtgtcatcgtgttgttagcagcagcggtgcagttggtggttatgaaggtggtctggcagttaaagaatggctgctggcacatgaaggccatcgtctgggcaaacctggtttaggcggtagcagtggtgcaagccctgcagcaccggctccggcaagtccagcagcaccagcacctagcgcaccagccggtggtaacattatgatgacccagagtccgagcagcctggcagttagtgccggtgaaaaagttaccatgacctgtaaaagcagccagagcgttctgtatagcagcaatcagaaaaactatctggcatggtatcagcagaaaccgggtcagagcccgaaactgctgatttattgggcaagcacccgtgaaagcggtgttccggatcgttttaccggtagcggtagcggcaccgattttaccctgaccattagcagcgttcagccggaagatctggcagtgtattattgtcatcagtatctgagcagccatacctttggtggtggcaccaaactggaaattaaacgcggtggtggtggtagtggtggcggtggttcaggcggtggcggtagccagctgcagcagccaggtgcagaactggttcgtccgggtagcagcgtgaaactgagctgtaaagcaagcggttatacctttacacgctattggattcattgggttaaacagcgtccgattcaaggtctggaatggattggtaatattgatccgagcgatagcgaaacccattacaaccagaaattcaaagataaagcaaccctgaccgtggataaaagcagtggcaccgcctatatgcagctgagcagtctgaccagcgaagatagcgcagtttattactgtgcaaccgaagatctgtattacgccatggaatattggggtcagggcaccagcgttaccgttagcagctaagtcgaccggctgctaacaaagcccgaaaggaagctgagttggctgctgccaccgctgagcaataactagcataaccccttggggcctctaaacgggtcttgaggggttttttgctactctggcgtcgatgaggga